(19) United States
(12) Patent Application Publication
(10) Pub. No.: US 2009/0130174 A1
(43) Pub. Date: May 21, 2009

(54) POLY (ESTER URETHANE) UREA FOAMS WITH ENHANCED MECHANICAL AND BIOLOGICAL PROPERTIES

(75) Inventors: Scott A. Guelcher, Franklin, TN (US); Andrea E, Hafeman, Nashville, TN (US); Lance I. Hochhauser, East Meadow, NY (US)

Correspondence Address: STITES & HARBISON PLLC, 401 COMMERCE STREET, SUITE 800, NASHVILLE, TN, 37219, US

(73) Assignee: Vanderbilt University

(21) Appl. No.: 12/195,265

(22) Filed: Aug. 20, 2008

Related U.S. Application Data
(60) Provisional application No. 60/956,897, filed on Aug. 20, 2007.

(51) Int. Cl. A61F 2/00; C08J 9/00; A61K 38/18; A61K 35/32; A61K 38/43; A61K 38/02; A61K 38/00; A61K 39/395; A61K 31/7088; A61K 31/70; A61K 35/00; A61K 35/76

(52) U.S. Cl. 424/426; 521/67; 514/12; 424/549; 424/94.1; 514/2; 514/8; 424/130.1; 514/44; 514/23; 424/93.1; 424/93.6

(57) ABSTRACT

A biodegradable polyurethane scaffold that includes a HDI trimer polyisocyanate and at least one polyol; wherein the density of said scaffold is from about 50 to about 250 kg m-3 and the porosity of the scaffold is greater than about 70 (vol %) and at least 50% of the pores are interconnected with another pore. The scaffolds of the present invention are injectable as polyurethane foams, and are useful in the field of tissue engineering.

### PRIORITY INFORMATION

This application claims benefit to U.S. Patent Application No. 60/956,897, filed Aug. 20, 2007, the contents of which are incorporated herein by reference in their entirety.

### GOVERNMENT RIGHTS

This invention was made with government support under US Army Institute of Surgical Research Grant No. W81XWH-06-0654, and W81XWH-07-1-0211. The government has certain rights in this invention.

### BACKGROUND OF THE INVENTION

Due to the high frequency of bone fractures, resulting in over 900,000 hospitalizations and 200,000 bone grafts each year in the United States, there is a compelling clinical need for improved fracture healing therapies. Fractures can result from trauma or pathologic conditions, such as osteoporotic compression fractures and osteolytic bone tumors. Autologous bone grafts are an ideal treatment due to their osteogenic, osteoinductive, and osteoconductive properties, but they are available in limited amounts and frequently result in donor site morbidity. Both synthetic and biological biomaterials have been investigated as substitutes for autogenous bone grafts, and a number of desirable properties have been identified for biomaterials designed for orthopedic applications. Their use can also be extended to soft tissue repair. The biomaterial and its degradation products must be biocompatible and non-cytotoxic, generating a minimal immune response. High porosity and inter-connected pores facilitate the permeation of nutrients and cells into the scaffold, as well as ingrowth of new tissue. Scaffolds should also undergo controlled degradation, preferably at a rate comparable to new tissue formation, to non-cytotoxic decomposition products. Materials that exhibit gel times of 5-10 minutes and low temperature exotherms are particularly suitable for clinical use as injectable therapies that can be administered percutaneously using minimally invasive surgical techniques. Additionally, scaffolds should possess sufficient biomechanical strength to withstand physiologically relevant forces. Release of growth factors with fibrogenic, angiogenic, and osteogenic properties, such as platelet-derived growth factor (PDGF), vascular endothelial growth factor (VEGF) and bone morphogenetic protein-2 (BMP-2), may further enhance integration of the device and improved healing.

Due to their ability to meet many of the above-mentioned performance characteristics, both synthetic and biopolymers have been investigated as scaffolds for tissue engineering. The poly(α-esters), including polylactic acid (PLA), polyglycolic acid (PGA), and their copolymers (PLGA), are thermoplastic polymers incorporated in a variety of FDA-approved biomedical devices, including surgical sutures, orthopedic fixation, and drug and growth factor delivery. Scaffolds prepared from other thermoplastic biomaterials, such as tyrosine-derived polycarbonates and polyphosphazenes, have been shown to exhibit tunable degradation to non-cytotoxic decomposition products, high tensile strength, and bone tissue ingrowth in vivo. However, thermoplastic biomaterials cannot be injected, and must be melt- or solvent-processed ex vivo to yield solid scaffolds prior to implantation. Injectable hydrogels, such as poly(ethylene glycol) (PEG), collagen, fibrin, chitosan, alginate, and hyaluronan, have been shown to support bone ingrowth in vivo, particularly when combined with angio-osteogenic growth factors. However, hydrogels lack the robust mechanical properties of thermoplastic polymers.

Two-component reactive polymers are promising scaffolds because they can be formed in situ without the use of solvents. Poly(propylene fumarate) (PPF) can be injected as a liquid and thermally or photo cross-linked in situ with various cross-linking agents, which affect the final mechanical and degradation properties. Recently developed porous composite scaffolds have been formed in situ by gas foaming, with up to 61% porosity, 50-500 μm pores, and a compressive modulus of 20-40 MPa. PPF biomaterials have been shown to support osteoblast attachment and proliferation in vitro, and ingrowth of new bone tissue in vivo. Growth factors have been incorporated via PLGA microspheres into poly(propylene fumarate) materials for controlled release.

Two-component biodegradable polyurethane (PUR) networks have also been investigated as scaffolds for tissue engineering. Porous PUR scaffolds prepared from lysine-derived and aliphatic polyisocyanates by reactive liquid molding have been reported to degrade to non-toxic decomposition products, while supporting the migration of cells and ingrowth of new tissue in vitro and in vivo. However, many polyisocyanates are toxic by inhalation, and therefore polyisocyanates with a high vapor pressure at room temperature, such as toluene diisocyanate (TDI, 0.018 mm Hg) and hexamethylene diisocyanate (HDI, 0.05 mm Hg), may not be suitable for injection in a clinical environment. To overcome this limitation, we and others have formulated injectable PUR biomaterials using lysine diisocyanate, a lysine-derived polyisocyanate with a vapor pressure substantially less than that of HDI. However, two-component polyurethanes prepared from LDI exhibit microphase-mixed behavior, which inhibits the formation of hydrogen bonds between hard segments in adjacent chains and may adversely affect mechanical properties.

In embodiments of the invention, porous scaffolds were synthesized by a one-shot foaming process, allowing for time to manipulate and inject the polymer, followed by rapid foaming and setting. Triisocyanate embodiments of the present invention exhibited superior characteristics related to biocompatibility, degradation, and mechanical properties were investigated. Additionally, the biodegradable PUR scaffolds of the present invention provide a vehicle for controlled release of growth factors was also examined. As anticipated, the PUR scaffolds synthesized from triisocyanates had elastomeric mechanical properties and substantially lower permanent deformation compared to LDI scaffolds. The reaction was mildly exothermic, such that the maximum temperature attained during foaming was 40° C. In vitro, the PUR scaffolds degraded hydrolytically on the order of months at a rate controlled by triisocyanate composition, while enzymatic and locally inflammatory activity seemed to accelerate in vivo degradation. All the PUR scaffolds exhibited both in vitro and in vivo biocompatibility, with minimal immune response limited to the material surface.

Thus, the present invention relates to biocompatible and biodegradable polymers. Particularly, the invention relates to biocompatible and biodegradable polyurethane foams. In several embodiments, the present invention relates to injectable polyurethane foams, to methods and compositions for their preparation and to the use of such foams as scaffolds for bone tissue engineering.

Synthetic biodegradable polymers are promising materials for bone tissue engineering. Many materials, including allografts, autografts, ceramics, polymers, and composites thereof are currently used as implants to repair damaged bone. Because of the risks of disease transmission and immunological response, the use of allograft bone is limited. Although autograft bone has the best capacity to stimulate healing of bone defects, explantation both introduces additional surgery pain and also risks donor-site morbidity. Synthetic polymers are advantageous because they can be designed with properties targeted for a given clinical application. Polymer scaffolds must support bone cell attachment, proliferation, and differentiation. Tuning the degradation rate with the rate of bone remodeling is an important consideration when selecting a synthetic polymer. Another important factor is the toxicity of the polymer and its degradation products. Furthermore, the polymer scaffold must be dimensionally and mechanically stable for a sufficient period of time to allow tissue ingrowth and bone remodeling.

Two-component reactive liquid polyurethanes designed for tissue repair have been disclosed. For example, U.S. Pat. No. 6,306,177, the disclosure of which is incorporate herein by reference, discloses a method for repairing a tissue site comprising the steps of providing a curable polyurethane composition, mixing the parts of the composition, and curing the composition in the tissue site wherein the composition is sufficiently flowable to permit injection by minimally invasive techniques and exhibits a tensile strength between 6,000 and 10,000 psi when cured. However, because this injectable polyurethane is non-porous and hard, tissue ingrowth is likely to be limited.

U.S. Pat. No. 6,376,742, the disclosure of which is incorporated herein by reference, discloses a method for in vivo tissue engineering comprising the steps of combining a flowable polymerizable composition including a blowing agent and delivering the resultant composition to a wound site via a minimally invasive surgical technique. U.S. Pat. No. 6,376,742 also discloses methods to prepare microcellular polyurethane implants as well as implants seeded with cells.

Bennett et al. prepared porous polyurethane implants for bone tissue engineering from isocyanate-terminated prepolymers, water, and a tertiary amine catalyst (diethylethanolamine). See, for example, Bennett S, Connolly K, Lee D R, Jiang Y, Buck D, Hollinger J O, Gruskin E A. Initial biocompatibility studies of a novel degradable polymeric bone substitute that hardens in situ. Bone 1996; 19(1, Supplement):101S-107S; U.S. Pat. Nos. 5,578,662, 6,207,767 and 6,339,130, the disclosures of which are incorporated herein by reference. The prepolymers were synthesized from lysine methyl ester diisocyanate (LDI) and poly(dioxanone-co-glycolide) from a pentaerythritol initiator and then combined with either hydroxyapatite or tricalcium phosphate to form a putty. Water and a tertiary amine were added to the putty prior to implantation in rats. The putty did not elicit an adverse tissue response following implantation.

Zhang et al. prepared biodegradable polyurethane foams from LDI, glucose, and poly(ethylene glycol). Zhang J, Doll B, Beckman E, Hollinger J O. A biodegradable polyurethane-ascorbic acid scaffold for bone tissue engineering. J. Biomed. Mater. Res. 2003; 67A(2):389-400; Zhang J, Doll B, Beckman J, Hollinger J O. Three-dimensional biocompatible ascorbic acid-containing scaffold for bone tissue engineering. Tissue Engineering 2003; 9(6):1 143-1157; Zhang J-Y, Beckman E J, Hu J, Yuang G-G, Agarwal S, Hollinger J O. Synthesis, biodegradability, and biocompatibility of lysine diisocyanate-glucose polymers. Tissue Engineering 2002; 8(5):771-785; and Zhang J-Y, Beckman E J, Piesco N J, Agarwal S. A new peptide-based urethane polymer: synthesis, biodegradation, and support of cell growth in vitro. Biomaterials 2000; 21:1247-1258., the disclosures of which are incorporated herein by reference. The foams were synthesized by reacting isocyanate-terminated prepolymers with water in the absence of catalysts. The polyurethane foams supported the attachment, proliferation, and differentiation of bone marrow stromal cells in vitro and were non-immunogenic in vivo. Bioactive foams were also prepared by adding ascorbic acid to the water prior to adding the prepolymer. As the polymer degraded, ascorbic acid was released to the matrix, resulting in enhanced expression of osteogenic markers such as alkaline phosphatase and Type I collagen.

Published PCT international patent application WO 2004/009227 A2, the disclosure of which is incorporated herein by reference, claims a star prepolymer composition suitable as an injectable biomaterial for tissue engineering. The prepolymer is the reaction product of a diisocyanate and a starter molecule having a molecular weight preferably less than 400 Da. Porous scaffolds were prepared by adding low levels (e.g., <0.5 parts per hundred parts polyol) of water.

Copending Published US Patent Application No. 2005/0013793 (U.S. patent application Ser. No. 10/759,904), the disclosure of which is incorporated herein by reference, discloses, inter alia, a biocompatible and biodegradable polyurethane composition including at least one biologically active component with an active hydrogen atom capable of reacting with isocyanates. As the polyurethane degrades in vivo, the bioactive component is released to the extracellular matrix where it is, for example, taken up by cells.

Published PCT international application WO 2006/055261, the disclosure of which is incorporated herein by reference, discloses a method of synthesizing of a biocompatible and biodegradable polyurethane foam includes the steps of: mixing at least one biocompatible polyol, water, at least one stabilizer, and at least one cell opener, to form a resin mix; contacting the resin mix with at least one polyisocyanate to form a reactive liquid mixture; and reacting the reactive liquid mixture form a polyurethane foam. The polyurethane foam is preferably biodegradable within a living organism to biocompatible degradation products. At least one biologically active molecule having at least one active hydrogen can be added to form the resin mix.

While materials such as those described above are useful for bone tissue engineering, it is desirable to improve certain properties associated with injectable polyurethane scaffolds. Highly porous (e.g., >80% or even >85%), fast-rising (e.g., <30 minutes) conventional polyurethane foams have been manufactured commercially for years. For example, Ferrari and co-workers' in Ferrari R J, Sinner J W, Bill J C, Brucksch W F. Compounding polyurethanes: Humid aging can be controlled by choosing the right intermediate. Ind. Eng. Chem. 1958; 50(7):1041-1044, and U.S. Pat. No. 6,066,681, the disclosures of which is incorporated herein by reference, disclose methods for preparation of polyurethane foams from diisocyanates and polyester polyols. Catalysts, including organometallic compounds and tertiary amines, are added to balance the gelling (reaction of isocyanate with polyol) and blowing (reaction of isocyanate with water) reactions. Stabilizer, such as polyethersiloxanes and sulfated castor oil, are added to both emulsify the raw materials and stabilize the rising bubbles. Cell openers, such as powdered divalent salts of stearic acid, cause a local disruption of the pore structure during the foaming process, thereby yielding foams with a natural sponge structure. See, for example, Oertel G., Polyurethane Handbook; and Berlin: Hanser Gardner Publications; 1994; Szycher, M, Szycher's Handbook of Polyurethanes, CRC Press, New York, N.Y., (1999), the disclosures of which are incorporated herein by reference.

However, conventional polyurethane foams are not suitable for tissue engineering applications because they are prepared from toxic raw materials, such as aromatic diisocyanates and organotin catalysts.

Although progress has been made in the development of biocompatible and biodegradable polymers, it remains desirable to develop biocompatible and biodegradable polymers, methods of synthesizing such polymers, implantable devices comprising such polymers and methods of using such polymers.

### SUMMARY OF THE INVENTION

In one aspect, the present invention provides a method of synthesizing of a biocompatible and biodegradable polyurethane foam including the steps of: mixing at least one biocompatible polyol, water, at least one stabilizer, and at least one cell opener, to form a resin mix; contacting the resin mix with at least one polyisocyanate to form a reactive liquid mixture; and reacting the reactive liquid mixture form a polyurethane foam. In embodiments, of the present invention, the polyisocyanate is a tri-functional isocyanate.

In other embodiments of the present invention, the resin mix comprises at least one biocompatible polyol, water, at least one stabilizer, at least one cell opener, and polyethylene glycol.

The polyurethane foam is preferably biodegradable within a living organism to biocompatible degradation products. At least one biologically active molecule having at least one active hydrogen can be added to form the resin mix.

To promote transport of cells, fluids, and signaling molecules, the foams can have a porosity greater than 50 vol-%. The porosity ε, or void fraction, is calculated as shown in WO '261 and below.

In several embodiments, at least one catalyst is added to form the resin mix. Preferably, the catalyst is non-toxic (in a concentration that may remain in the polymer).

The catalyst can, for example, be present in the resin mix in a concentration in the range of approximately 0.5 to 6 parts per hundred parts polyol and, preferably in the range of approximately 1 to 5. The catalyst also can, for example, be an organometallic compound or a tertiary amine compound. In several embodiments the catalyst includes stannous octoate, an organobismuth compound, triethylene diamine, bis(dimethylaininoethyl)ether, or dimethylethanolamine. An example of a preferred catalyst is triethylene diamine.

In several embodiments, the polyol is biocompatible and has a hydroxyl number in the range of approximately 50 to 1600. The polyol can, for example, be a biocompatible and polyether polyol or a biocompatible polyester polyol. In several embodiments, the polyol is a polyester polyol synthesized from at least one of ε-caprolactone, glycolide, or DL-lactide.

Water can, for example, be present in the resin mix in a concentration in a range of approximately 0.1 to 4 parts per hundred parts polyol.

The stabilizer is preferably nontoxic (in a concentration remaining in the polyurethane foam) and can include non-ionic surfactant or an anionic surfactant. The stabilizer can, for example, be a polyethersiloxane, a salt of a fatty sulfonic acid or a salt of a fatty acid, in the case that the stabilizer is a polyethersiloxane, the concentration of polyethersiloxane in the resin mix can, for example, be in the range of approximately 0.25 to 4 parts per hundred polyol. In the case that the stabilizer is a salt of a fatty sulfonic acid, the concentration of the salt of the fatty sulfonic acid in the resin mix is in the range of approximately 0.5 to 5 parts per hundred polyol. In the case that the stabilizer is a salt of a fatty acid, the concentration of the salt of the fatty acid in the resin mix is in the range of approximately 0.5 to 5 parts per hundred polyol. Polyethersiloxane stabilizers are preferably hydrolyzable. Examples of suitable stabilizers include a sulfated castor oil or sodium ricinoleicsulfonate.

The cell opener is preferably nontoxic (in a concentration remaining in the polyurethane) and comprises a divalent metal salt of a long-chain fatty acid having from about 1-22 carbon atoms. The cell opener can, for example, include a metal salt of stearic acid. The concentration of the cell opener in the resin mix is preferably in the range of approximately 0.5 to 7 parts per hundred polyol.

The polyisocyanate can, for example, be a biocompatible aliphatic polyisocyanate derived from a biocompatible polyamine compound (for example, amino acids). Examples of suitable aliphatic polyisocyanates include lysine methyl ester diisocyanate, lysine triisocyanate, 1,4-diisocyanatobutane, or hexamethylene diisocyanate. As stated above, embodiments of the present invention comprises tri-functional isocyanate.

The index of the foam, as defined by:

INDEX=100×number of NCO equivalents/number of OH equivalents and can be in the range of approximately 80 to 140.

The polyurethane foams of the present invention are preferably synthesized without aromatic isocyanate compounds. The method of the present invention can also include the step of placing the reactive liquid mixture in a mold in which the reactive liquid mixture is reacted to form the polyurethane foam.

In another aspect, the present invention provides a biocompatible and biodegradable polyurethane synthesized via the steps of: mixing at least one polyol, PEG, water, at least one stabilizer, and at least one cell opener; contacting the resin mix with at least one triisocyanate to form a reactive liquid mixture; and reacting the reactive liquid mixture to form a polyurethane foam. The polyurethane foam is preferably biodegradable within a living organism to biocompatible degradation products. At least one catalyst, as described above, can be added to form the resin mix. As also described above, at least one biologically active molecule having at least one active hydrogen can be added to form the resin mix.

In another aspect, the present invention provides method of synthesis of a biocompatible and biodegradable polyurethane foam including the steps of: reacting at least one polyol and PEG with at least one triisocyanate to form an isocyanate-terminated prepolymer; mixing water, at least one stabilizer, at least one cell opener and at least one polyol to form a resin mix; contacting the resin mix with the prepolymer to form a reactive liquid mixture; and reacting the reactive liquid mixture to form a polyurethane foam. At least one catalyst, as described above, can be added to form the resin mix. As also described above, at least one biologically active molecule having at least one active hydrogen can be added to form the resin mix.

The invention can, for example, provide dimensionally stable, high porosity, injectable, biocompatible, biodegradable and (optionally) biologically active polyurethane foams. The open-pore content can be sufficiently high to prevent shrinkage of the foam. The foams of the present invention can, for example, support the attachment and proliferation of cells in vitro and are designed to degrade to and release biocompatible components in vivo. In that regard, the present invention also provides scaffolds for cell proliferation/growth comprising a polyurethane polymer as set forth above and/or fabricated using a synthetic method as described above.

Typically, the biodegradable compounds of the present invention degrade by hydrolysis. As used herein, the term “biocompatible” refers to compounds that do not produce a toxic, injurious, or immunological response to living tissue (or to compounds that produce only an insubstantial toxic, injurious, or immunological response). The term nontoxic as used herein generally refers to substances or concentrations of substances that do not cause, either acutely or chronically, substantial damage to living tissue, impairment of the central nervous system, severe illness, or death. Components can be incorporated in nontoxic concentrations innocuously and without harm. As used herein, the term “biodegradable” refers generally to the capability of being broken down in the normal functioning of living organisms/tissue (preferably, into innocuous, nontoxic or biocompatible products).

The polyurethanes compositions of the present invention are useful for a variety of applications, including, but not limited to, injectable scaffolds for bone tissue engineering and drug and gene delivery. The compositions of the present invention can, for example, be applied to a surface of a bone, deposited in a cavity or hole formed in a bone, injected into a bone or positioned between two pieces of bone. The compositions can be injected through the skin of a patient to, for example, fill a void, cavity or hole in a bone using, for example, a syringe. Likewise, the compositions of the present invention can be molded into any number of forms outside of the body and placed into the body. For example, the compositions of the present invention can be formed into a plate, a screw, a prosthetic element, a molded implant etc.

The invention encompasses methods and compositions for preparing biocompatible and biodegradable polyurethane foams that are dimensionally stable.

One embodiment of the present invention is a method of synthesizing of a biocompatible and biodegradable polyurethane foam comprising the steps of: mixing at least one biocompatible polyol, PEG, water, at least one stabilizer, and at least one pore opener, to form a resin mix; contacting the resin mix with at least one HDIt polyisocyanate to form a reactive liquid mixture; and reacting the reactive liquid mixture form a polyurethane foam. In this embodiment, the polyurethane foam being biodegradable within a living organism to biocompatible degradation products. In other aspects of this embodiment, at least one catalyst is added to form the resin mix.

The PEG may have a MW of 600, for example. The PEG may be added in an amount up to about 60% polyol component.

In other embodiments of the present invention, the mixing step comprises mixing a catalyst, stabilizer, and pore opener. The catalyst may be a triethylenediamine catalyst. The stabilizer may be a sulfated castor oil stabilizer. The pore opener may be a calcium stearate cell opener.

Another embodiment of the present invention is a biodegradable polyurethane scaffold, comprising a HDI trimer polyisocyanate and at least one polyol; wherein the density of said scaffold is from about 50 to about 250 kg m-3 and the porosity of the scaffold is greater than about 70 (vol %) and at least 50% of the pores are interconnected with another pore.

The density of this embodiment may be at least 90 kg m-3. In other aspects, the density may be at least from about 75 to about 125 kg m-3.

Aspects of this embodiment may further comprise PEG. The PEG may be present in an amount of about 50% or less w/w. In other aspects, the PEG may be present in an amount of about 30% or less w/w.

In scaffolds of the present invention, the glass transition temperature may be in a range of about −50 to about 20. In other aspects of the present invention, the glass transition temperature is in a range of about −20 to about 10.

The porosity of the polyurethane scaffolds of the present invention may be, for example, greater than 70 (vol-%). In other aspects, the porosity may be from about 90 to about 95 (vol-%).

The pore size of scaffolds of the present invention may be, for example, about 100-1000 μm. In other aspects, the pore size may be about 200-500 μm.

The polyurethane scaffolds of the present invention may be comprised of at least one growth factor. Examples of the growth factors are PDGF, VEGF, and BMP-2.

The polyurethane scaffolds of the present invention may optionally further comprise a stabilizer, such as a stabilizer chosen from a polyethersiloxane, sulfonated caster oil, and sodium ricinoleicsulfonate.

The polyurethane scaffolds of the present invention may further comprise a biologically active agent. One example of a biologically active agent is demineralized bone particles. Other examples include agents chosen from enzymes, organic catalysts, ribozymes, organometallics, proteins, glycoproteins, peptides, polyamino acids, antibodies, nucleic acids, steroidal molecules, antibiotics, antivirals, antimycotics, anticancer agents, analgesic agents, antirejection agents, immunosuppressants, cytokines, carbohydrates, oleophobics, lipids, extracellular matrix and/or its individual components, demineralized bone matrix, pharmaceuticals, chemotherapeutics, cells, viruses, virenos, virus vectors, and prions.

In aspects of the present invention, the HDI trimer may be present in an amount of from about 30 to about 75 wt %. In other aspects, the HDI trimer is present in an amount of from about 40 to about 70 wt %.

In aspects of the present invention, the polyol is a polyester triol present in an amount of from about 10 to about 70 wt %. In other aspects, polyol is a polyester triol present in an amount of from about 20 to about 60 wt %.

In other aspects of the present invention, the PEG may be present in an amount of about 40 wt % or less. In others, the PEG is present in an amount of about 30 wt % or less.

Additionally, in aspects of the present invention, the polyurethane scaffolds have a permanent deformation of the scaffold is less than about 3.0%.

In another embodiment of the present invention, included is a biodegradable polyurethane scaffold that comprises a HDI trimer polyisocyanate in an amount of from about 40 to about 70 wt %, a polyester triol present in an amount of from about 20 to about 60 wt %, and PEG in an amount of from about 30 wt % or less; wherein the permanent deformation of the scaffold is less than about 3.0%.

The present invention, along with the attributes and attendant advantages thereof, will best be appreciated and understood in view of the following detailed description taken in conjunction with the accompanying drawings.

### BRIEF DESCRIPTION OF THE DRAWINGS

FIG. 1 shows the chemical structures of lysine diisocyanate (LDI), lysine triisocyanate (LTI), and hexamethylene diisocyanate trimer (HDIt).

FIG. 2 shows compression set of LTI, HDIt, HDIt+50% PEG, and LDI scaffolds made with the 900-Da triol. LDI materials had larger permanent deformations than did materials with either of the triisocyanates.

FIG. 3 shows SEM images of foams made with the 900-Da triol suggest interconnected pore structures with mostly uniform pore sizes of 200-1000 μm. a) LTI (scale bar 600 μm), b) HDIt (scale bar 600 μm), C) HDIt+50% PEG (scale bar 750 μm).

FIG. 4 demonstrates the injectability of examples of PUR scaffolds of the present invention, and includes time-lapse photographs showing injection of the reactive liquid system.

FIG. 5 shows in vitro degradation of PUR scaffolds. At 36 weeks, both LTI materials had completely degraded, while the HDIt materials remained at 52-81% of their original masses. Although PEG initially accelerated degradation within the first 4 weeks, it slowed the long-term degradation rates.

FIG. 6 shows storage and loss moduli as a function of shear rate during DMA frequency sweeps from 0.1 to 10 Hz, and stress relaxation response to 2% strain over 20 minutes. Panels are shown in order of increasing T_{g} (left to right): materials with PEG (a & d), with 1800-Da polyol (b & e), and 900-Da polyol (c & f).

FIG. 7 shows stress-strain curves measured in compression mode. Young's Modulus values were calculated from the initial slopes.

FIG. 8 shows calcein AM staining of live cells (green) seeded on PUR scaffolds, which autofluoresce red (excitation/emission 495/515 nm). a) LTI, b) HDIt, c) HDIt+50% PEG.

FIG. 9 shows trichrome stain of subcutaneous in vivo implants after 5, 14, and 21 days. All scaffolds shown were made with the 900-Da triol. Material remnants are shown as white segments. Granulation tissue, collagen deposition, and giant cell response are visible.

FIG. 10 shows in vitro release of lyophilized ¹²⁵I-PDGF (10 μg and 50 μg per g of scaffold) from T6C3G1L900/HDIt+50% PEG scaffold. Cumulative release expressed as percentage of total ¹²⁵I-PDGF initially contained in sample.

### DETAILED DESCRIPTION OF THE INVENTION

One embodiment of a reactive liquid molding process of the present invention for preparing the polyurethane foam is contacting an aliphatic polyisocyanate (or an isocyanate-terminated prepolymer) component (component 1) with a resin mix component (component 2) comprising at least one polyol, PEG, water, and optionally at least one cell opener. Preferably at least one catalyst is also present in the resin mix component. In several embodiments, one or more bioactive components are present in the resin mix component. The resin mix of component 2 is mixed with the polyisocyanate or multi-functional isocyanate compounds (that, compounds have a plurality of isocyanate function groups) of component 1 to form a reactive liquid composition. The reactive liquid composition can, for example, be cast into a mold either inside or outside the body where it cures to form a porous polyurethane. Thus, as used herein the term “mold” refers generally to any cavity or volume in which the reactive liquid composition is placed, whether that cavity or volume is formed manually or naturally outside of a body or within a body. See, for example, WO 2006/055,261.

The polyisocyanate reacts with compounds in the resin mix having an active hydrogen (e.g., polyol and water). Useful polyisocyanates include aliphatic polyisocyanates, such as lysine methyl ester diisocyanate (LDI), lysine triisocyanate (LTI), 1,4-diisocyanatobutane (BDI), and hexamethylene diisocyanate (HDI), and dimers and trimers of HDI. HDI trimer and LTI are examples of preferred polyisocyanates for use in the present invention.

In embodiments of the invention, the value of the index is in the range of approximately 80 to 140 and, more preferably, in the range of approximately 100 to 130.

In embodiments of the present invention, the hydroxyl number of the polyol/polyol blend is in the range of approximately 50 to 1600.

Polyester polyols are particularly suitable for use in the present invention because they hydrolyze in vivo to non-toxic, biocompatible degradation products. In several preferred embodiments of the present invention, the polyol is a polyester polyol or blend thereof having a hydroxyl number preferably in the range of approximately 80 to 420. Polyester polyols suitable for use in the present invention can, for example, be synthesized from at least one of the group of monomers including ε-caprolactone, glycolide, or DL-lactide.

Water reacts with polyisocyanate to form a disubstituted urea and carbon dioxide, which acts as a blowing agent. This reaction is referred to as the blowing reaction and results in a porous structure. The concentration of water in the resin mix affects the porosity and pore size distribution. To promote the presence of inter-connected pores, the concentration of water in the resin mix is preferably in the range of approximately 0.1 to 5 parts per hundred parts polyol (pphp) and, more preferably, in the range of approximately 0.5 to 3 pphp.

To form a dimensionally stable and highly porous foam, the rates of the gelling and blowing reactions are preferably balanced. This balance of rates can be accomplished through the use of catalysts, which can, for example, include an organometallic urethane catalyst, a tertiary amine urethane catalyst or a mixture thereof. In general, suitable catalysts for use in the present invention include compounds known in the art as effective urethane blowing and gelling catalysts, including, but not limited to, stannous octoate, organobismuth compounds (e.g., Coscat 83), triethylene diamine, bis(dimethylaminoethyl)ether, and dimethylethanolamine. Tertiary amine catalysts are preferred as a result of their generally lower toxicity relative to, for example, organometallic compounds. Triethylene diamine, which functions as both a blowing and gelling catalyst, is particularly preferred. Concentrations of catalyst blend in the resin mix are preferably in the range or approximately 0.1 to 6 pphp and, more preferably, in the range of approximately 0.5 to 5.0 pphp and, even more preferably, in the range of approximately 1 to 5 or in the range of approximately 1 to 4.

Foam stabilizers can be added to the resin mix of the present invention to, for example, disperse the raw materials, stabilize the rising carbon dioxide bubbles, and/or control the pore size of the foam. Although there has been a great deal of study of foam stabilizers (sometimes referred to herein as simply “stabilizers”) the operation of stabilizers during foaming is not completely understood. Without limitation to any mechanism of operation, it is believed that stabilizers preserve the thermodynamically unstable state of a foam during the time of rising by surface forces until the foam is hardened. In that regard, foam stabilizers lower the surface tension of the mixture of raw materials and operate as emulsifiers for the system. Stabilizers, catalysts and other polyurethane reaction components are discussed, for example, in Oertel, Günter, ed., Polyurethane Handbook, Hanser Gardner Publications, Inc. Cincinnati, Ohio, 99-108 (1994). A specific effect of stabilizers is believed to be the formation of surfactant monolayers at the interface of higher viscosity of the bulk phase, thereby increasing the elasticity of the surface and stabilizing expanding foam bubbles.

Stabilizers suitable for use in the present invention include, but are not limited to, non-ionic surfactants (e.g., polyethersiloxanes) and anionic surfactants (e.g., sodium or ammonium salts of fatty sulfonic acids or fatty acids). Polyethersiloxanes, sulfated castor oil (Turkey red oil), and sodium ricinoleicsulfonate are examples of preferred stabilizers for use in the present invention. In the case of polyethersiloxane stabilizers, the concentrations of polyethersiloxane stabilizer in the resin mix is preferably in the range of approximately 0.25 to 4 pphp and, more preferably, in the range of approximately 0.5 to 3 pphp. Preferably, polyethersiloxane compounds for use in the present invention are hydrolyzable. In the case of stabilizers including salts of fatty sulfonic acid and/or salts of fatty acid, the concentration of salts of a fatty sulfonic acid and/or salts of a fatty acid in the resin mix is preferably in the range of approximately 0.5 to 5 pphp and, more preferably, in the range of approximately 1 to 3 pphp.

Cell openers or cell opening agents can be added to the resin mix to, for example, disrupt the pore structure during the foaming process, thereby creating foams with a natural sponge structure. Cell openers reduce the tightness and shrinkage of the foam, resulting in dimensionally stable foams with inter-connected pores. Cell openers and other reaction components of polyurethane foams are discussed, for example in Szycher, M, Szycher's Handbook of Polyurethanes, CRC Press, New York, N.Y., 9-6 to 9-8 (1999). Cell openers suitable for use in the present invention include powdered divalent metal salts of long-chain fatty acids having from about 1-22 carbon atoms. Divalent metal salts of stearic acid, such as calcium and magnesium stearate, are examples of preferred cell openers for use in the present invention. The concentrations of cell openers in the resin mix is preferably in the range of approximately 0.5-7.0 pphp and, more preferably, in the range of approximately 1 to 6 pphp.

Biologically active agents can optionally be added to the resin mix. As used herein, the term “bioactive” refers generally to an agent, a molecule, or a compound that affects biological or chemical events in a host. Bioactive agents may be synthetic molecules, biomolecules, or multimolecular entities and include, but are not limited to, enzymes, organic catalysts, ribozymes, organometallics, proteins, glycoproteins, peptides, polyamino acids, antibodies, nucleic acids, steroidal molecules, antibiotics, antivirals, antimycotics, anticancer agents, analgesic agents, antirejection agents, immunosuppressants, cytokines, carbohydrates, oleophobics, lipids, extracellular matrix and/or its individual components, demineralized bone matrix, pharmaceuticals, chemotherapeutics, and therapeutics. Cells and non-cellular biological entities, such as viruses, virenos, virus vectors, and prions can also be bioactive agents. Biologically active agents with at least one active hydrogen are preferred. Examples of chemical moieties with an active hydrogen are amine and hydroxyl groups. The active hydrogen reacts with free isocyanate in the reactive liquid mixture to form a covalent bond (e.g., urethane or urea linkage) between the bioactive molecule and the polyurethane. As the polyurethane degrades, the bioactive molecules are released and are free to elicit or modulate biological activity. The incorporation of biologically active components into biocompatible and biodegradable polyurethanes is discussed in some detail in US Patent Application No. 2005/0013793 (U.S. patent application Ser. No. 10/759,904).

After mixing the polyisocyanate and the resin mix, the resulting reactive liquid mixture is, for example, cast into a cavity or mold where the polyisocyanate reacts with the components of the resin mix having an active hydrogen to form a polyurethane foam. The reactive liquid mixture can be cast into a mold ex vivo and then implanted or can be cast directly onto a surface or into a cavity, volume or mold (for example, a wound) in the body.

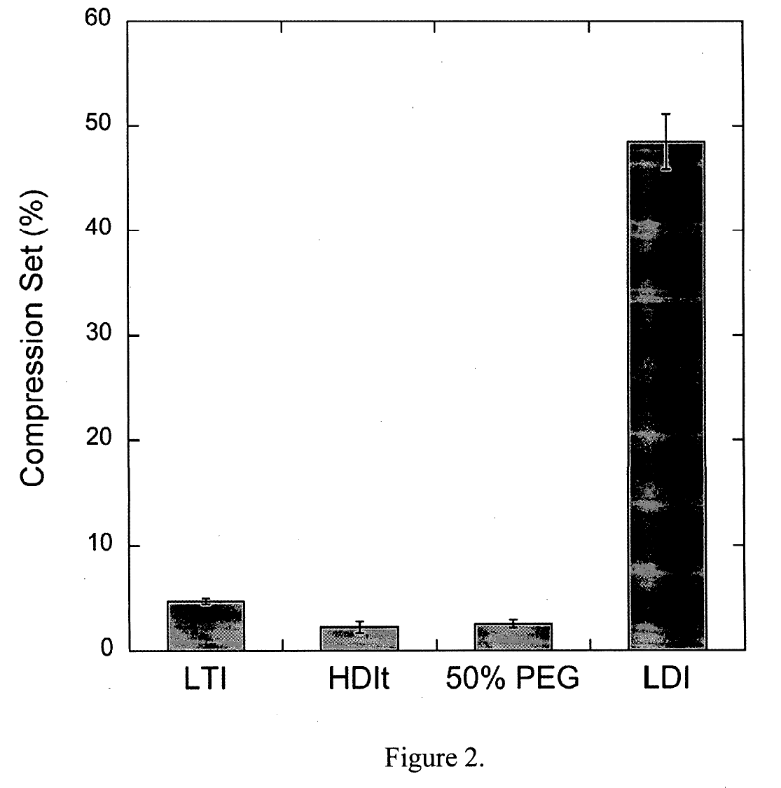

With respect to PUR scaffold characterization features of the present invention, the permanent deformation, or compression set, of LDI, LTI, HDIt, and HDIt+50% PEG examples of the present invention is shown in the FIG. 2.

The LTI (4.7±0.3%), HDIt (2.2±0.5%), and HDIt+50% PEG (2.5±0.5%) embodiments of the present invention exhibited minimal permanent deformation after being subjected to a 50% compressive strain for 24 hours. In contrast, materials synthesized from lysine methyl diisocyanate (LDI) displayed a substantially higher compression set of 48.5±2.6%, with statistically significant differences (p<0.005). Thus the PUR scaffolds synthesized from triisocyanates were more resilient than those prepared from diisocyanates. The favorable mechanical properties of segmented polyurethane elastomers and foams have been attributed to microphase-separation of hard and soft segments and subsequent hydrogen bonding between hard segments. However, previous studies have shown that PUR scaffolds prepared from LDI were microphase-mixed and exhibited negligible hydrogen bonding between adjacent hard segments due to the asymmetric structure of LDI. Similarly, the FT-IR spectra for LTI and HDIt scaffolds also suggested minimal hydrogen-bonded urethane and urea groups (data not shown). The substantially lower compression set observed for PUR networks synthesized from triisocyanates is thus conjectured to result from the greater degree of chemical crosslinking relative to that achieved with diisocyanates.

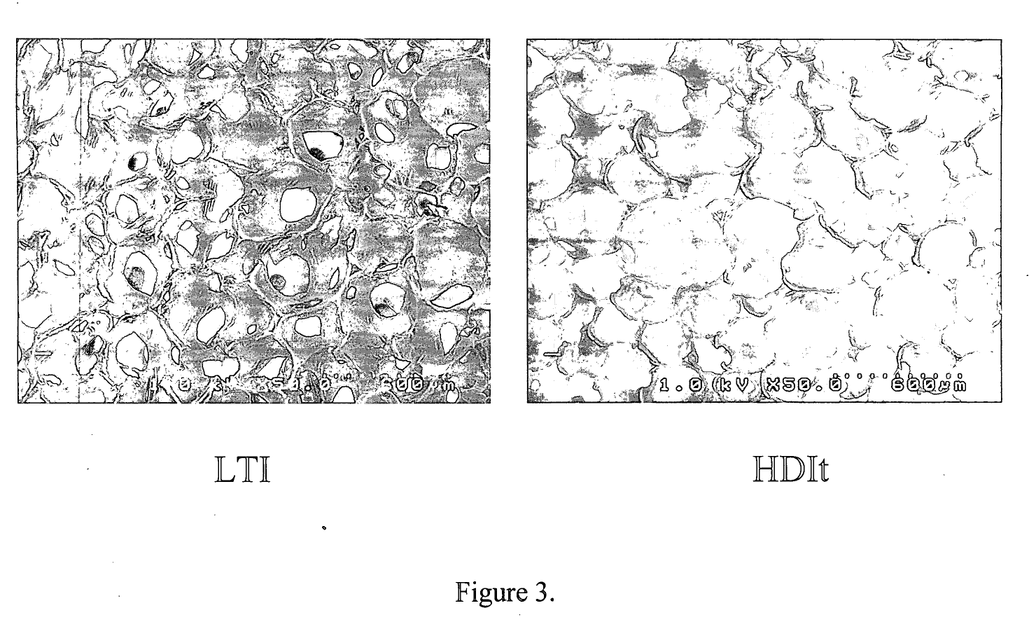

The core densities and porosities of the scaffolds (Table 1, below) were assessed at least 24 hours after foam synthesis to ensure full curing and drying. The density of the scaffolds ranged from 86-98 kg m⁻³ and the porosity from 92-93 vol %. The differences between the densities and porosities measured for the materials were not statistically significant (p>0.05). SEM images (FIG. 3) illustrated that the pores were almost uniformly spherical, 200-400 μm in diameter, and inter-connected by openings in the pore walls. Previous studies with LDI scaffolds have shown that MC3T3 cells penetrated up to 5 mm into the interior of the scaffolds after 21 days, suggesting that the pores were inter-connected. See, for example, S. Guelcher, A. Srinivasan, A. Hafeman, K. Gallagher, J. Doctor, S. Khetan, S. McBride, and J. Hollinger. Synthesis, in vitro degradation, and mechanical properties of two-component poly(ester urethane)urea scaffolds: Effects of water and polyol composition. Tissue Engineering 13: 2321-2333 (2007).

Addition of PEG had an insignificant effect on the scaffold density and porosity, but SEM showed that the pores were more irregularly shaped and variable in size, reaching 600 μm in diameter. The irregular pore shape and rough surface are thought to result from phase-separation of the PEG and polyester polyol components.

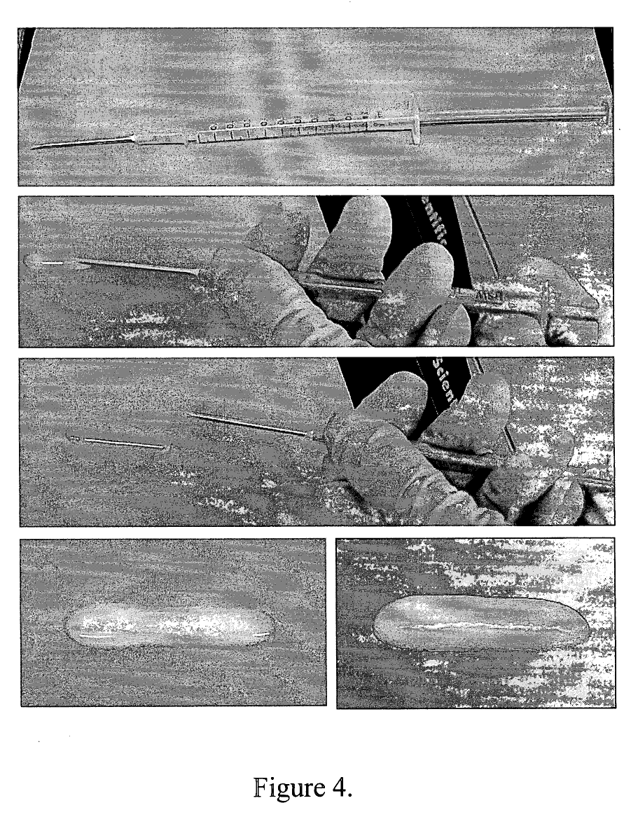

A significant advantage of the PUR scaffolds is that they are injectable, as shown in FIG. 4, and therefore should have capability of being administered using minimally invasive surgical techniques. With respect to the reaction temperatures of injectable systems, the reaction of polyester polyol and isocyanate to form urethane bonds is exothermic, although the aliphatic polyisocyanates used in this study are less reactive than aromatic polyisocyanates. The maximum temperature in the center of the foam was 30.5° C. for HDIt materials and 40.0° C. for LTI materials, both of which are significantly lower than typical exotherm temperatures of up to 110° C. for poly(methyl methacrylate) (PMMA). The gel times of the mixtures, estimated by observing the change in viscosity from a viscous liquid to a non-flowable gel, were approximately 3 minutes (LTI) and 5 minutes (HDIt). Despite the higher catalyst concentration used in the HDIt formulations, these polymers exhibited lower reaction exotherms and longer gel times, suggesting that HDIt is less reactive than LTI.

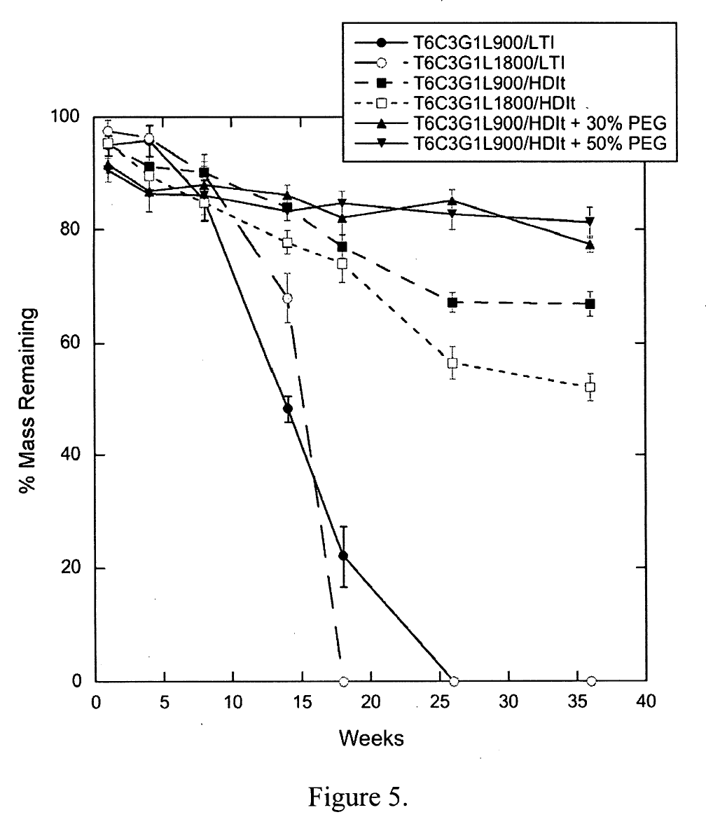

The degradation rates are shown in FIG. 5. All of the materials retained 85-90% of their original mass after 8 weeks. The LTI scaffolds degraded rather quickly thereafter, with only 22% (900/LTI) and 48% (1800/LTI) mass remaining after 14 and 18 weeks, respectively, and no intact mass remaining by 36 weeks. On the other hand, the HDIt materials degraded steadily, with 52-81% mass remaining at 36 weeks. Although PEG initially accelerated degradation within the first 4 weeks, it slowed the long-term degradation rates.

Polyurethane scaffolds synthesized from aliphatic and lysine-derived polyisocyanates have been reported to support cell attachment and proliferation in vitro, as well as ingrowth of new tissue and degradation to non-cytotoxic decomposition products in vivo. While the low vapor pressure of LDI renders it useful for injectable biomaterials, LDI-based PUR scaffolds synthesized by the gas foaming process displayed poor resiliency, with up to 50% permanent deformation when subjected to compressive loads. The high compression set of LDI-based PUR scaffolds is conjectured to result from the absence of physical crosslinks in the polymer network, as evidenced by the lack of hydrogen-bonded urethane and urea groups in the hard segment. For segmented PUR elastomers synthesized from LDI, the microphase morphology depends on the molecular weight of the soft segment. For LDI elastomers incorporating a 2000 g mol⁻¹ poly(ε-caprolactone) (PCL) diol soft segment, the value of T_{g} was −52° C., which is close to that of pure PCL diol. However, for soft segments with molecular weights of 1250 or 530 g mol⁻¹, the value of T_{g} increased 20-45° C., suggesting the presence of significant microphase-mixing that has been attributed to the asymmetric ethyl branch in LDI. Considering that microphase-mixing of LDI segmented elastomers becomes significant at soft segment equivalent weights of 625 g eq⁻¹, it is not surprising that LDI-based PUR networks exhibited microphase-mixing at soft segment equivalent weights of 300 g eq⁻¹. We reasoned that triisocyanates would yield PUR networks with higher chemical crosslink density, thus compensating for the lack of physical crosslinks and improving mechanical properties such as compression set. In this study, PUR scaffolds were prepared from LTI and Desmodur N3300A HDI trimer using the one-shot gas foaming process as described previously for LDI. Both HDIt and LTI have low vapor pressure at ambient temperature, thus minimizing the risk of exposure by inhalation when the materials are injected. Furthermore, it was of interest to compare the biocompatibility and degradation of PUR scaffolds synthesized from aliphatic and lysine-derived triisocyanates. While LTI and HDIt have been used to synthesize cast elastomers with improved properties, such as optical clarity and thermal stability, their use in biodegradable PUR scaffolds has not been previously reported. The effects of triisocyanate composition on biocompatibility, biodegradation, and mechanical properties were investigated, as well as the use of the PUR scaffolds for release of growth factors.

The data in FIG. 2 demonstrate that the PUR networks synthesized from LTI and HDIt exhibited significantly lower permanent deformation than those synthesized from LDI. Materials in wound healing applications could benefit from greater resilience, which would allow them to better conform to the wound site and maintain contact with the host tissue when subjected to compressive or tensile forces.

Polyether and polyester polyols have been mixed in previous studies to produce foams via prepolymers and chain extension, but not for one-shot foams prepared directly from polyisocyanates without the prepolymer step. Polyethers are generally immiscible with polyesters and are typically stabilized with water-soluble polyethersiloxanes. However, foams with polyethersiloxane stabilizers have been reported not to support cell attachment or proliferation. Instead, we have shown that stable scaffolds can be synthesized with polyether-polyester mixtures using turkey red oil as a stabilizer and surfactant as previously used to stabilize polyester foams. These materials were stable with up to 70% PEG.

As shown in Table 1, the composition of the polyol component had a substantial effect on the glass transition temperatures of the PUR scaffolds. PUR scaffolds prepared from the 1800 g mol⁻¹ (600 g eq⁻¹) polyol had T_{g} values ˜20° C. higher than those prepared from 900 g mol⁻¹ (300 g eq⁻¹) polyol, which is consistent with the effects of soft segment equivalent weight on T_{g} observed previously for segmented PUR elastomers prepared from LDI. The addition of PEG also reduced the T_{g} of the PUR networks, which is attributed to the lower T_{g} of PEG relative to the polyester polyols. As anticipated, the PUR networks did not display any melting transitions because amorphous polyols were used. In a previous study, PUR scaffolds synthesized from HDIt with PEG and poly(ε-caprolactone) polyols exhibited melting transitions (associated with the semi-crystalline soft segments) ranging from 39-58° C. (44). However, no glass transitions were reported within the range of −20-200° C., so the extent of microphase separation of the materials is not known.

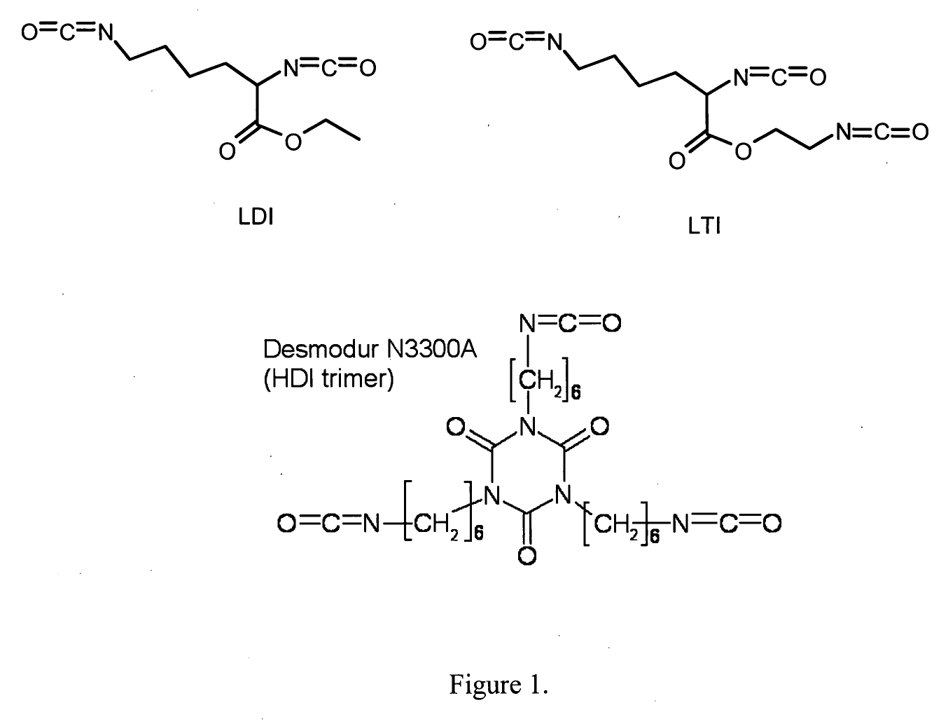

While previous studies showed that in vitro degradation is controlled by the polyol composition, the data in FIG. 5 demonstrate that the polyisocyanate composition also has a dramatic effect on the degradation of the PUR scaffolds. LTI scaffolds degraded faster than the HDIt materials, which has been attributed to the degradable ester linkage present in the backbone of lysine derived polyisocyanates (see FIGS. 1a and b). Hydrolysis of this ester group yields a carboxylic acid group in the polymer, which has been suggested to catalyze further degradation. For lysine-derived polyisocyanates, hydrolysis of urethane linkages to lysine has been reported, while others have reported that urethane and urea linkages are only enzymatically degraded. Higher soft segment content may also explain the faster degradation of the LTI materials, due to the higher % NCO (lower equivalent weight) of LTI relative to that of HDIt. In vivo, the materials degraded significantly faster than in vitro, an observation that has been documented previously for porous poly(D-lactic-co-glycolic acid) scaffolds and most likely due to an enzymatic mechanism. Furthermore, enzymatic cleavage of the lysine residues likely contributes to accelerated degradation of the LTI scaffolds in vivo. Previous studies have shown that the addition of PEG increases the hydrolytic degradation rate, presumably due to the increased hydrophilicity with PEG. The addition of PEG 600 to HDIt foams increased the initial degradation rate (1-8 weeks), which is attributed to increased bulk hydrophilicity resulting from higher PEG content. This increases water absorption into the material, which results in enhanced diffusion of water to hydrolyze the ester linkages, and faster diffusion of degradation products out of the scaffold. However, at later time points (10-36 weeks), the degradation rate decreased, which is inconsistent with previous studies. Furthermore, addition of PEG was observed to increase the polymer degradation in vivo in the subcutaneous implant model. The cause of the discrepancy between the in vitro and in vivo degradation data is not known.

The PUR scaffolds exhibited elastomeric dynamic mechanical properties, as evidenced by their high elongation at break and low compression set; they ranged from ideal elastomers, where the deformation energy is primarily stored elastically, to high-damping elastomers, where the energy is both stored elastically and thermally dissipated. By varying the composition of the triisocyanate and polyol components, it is possible to prepare elastomeric PUR scaffolds with tunable damping properties. Application of rubbery elastomers (i.e., low-damping) as scaffolds for bone defects has been suggested to promote intimate contact between the implant and the host bone. The elastomer can be compressed prior to implantation, where it then expands in the wound to maintain intimate contact with the local tissue. Maintaining good contact between the bone and implant may promote the migration of local osteoprogenitor cells from the bone into the implant, thereby enhancing bone regeneration. It has also been suggested that elastomeric properties can protect the implant from shear forces at the bone-implant interface. However, the effects of the damping properties of the scaffold on tissue regeneration are not known. If the damping is excessive, then, upon exposure to physiological strains, the relaxation modulus may drop to values too low to provide significant support.

Materials prepared from triisocyanates in the present study displayed slightly higher densities but comparable porosities to one-shot polyurethanes made from LDI in a previous study. However, the compressive strength (i.e., the compressive stress measured at 50% strain) of the HDIt and LTI materials (5-15 kPa) was higher than that of the LDI materials (2-4 kPa). HDI-prepolymer foams of comparable density (80-107 kg m⁻³) from a previous study were generally stronger than the one-shot HDIt and LTI foams of the present study, with compressive strengths of 30-85 kPa (at 40% strain) versus 5-15 kPa (at 50% strain) for the HDIt and LTI foams. However, the Young's moduli of the HDI-prepolymer foams are lower, at 9-21 kPa, compared to 26-202 kPa for the one-shot foams. While the HDI-prepolymer foams exhibit elastomeric mechanical properties and good biocompatibility in vivo, they are not injectable due to the high temperature (60° C.) cure step.

In a previous study, endothelial cell adhesion in vitro to a poly(ether ester urethane)urea scaffold was inversely proportional to the hydrophilicity, although smooth muscle cells grew faster in the more hydrophilic scaffold. Bone regeneration occurred in polyurethane scaffolds implanted into defects of sheep iliac crests, with more calcium phosphate salts mineralized in defects with hydrophilic scaffolds, which also had the highest porosities (43). Original ilium thickness was reestablished only in defects with the most hydrophobic scaffolds, perhaps because their slow degradation rates allowed more time for bone ingrowth. In the PUR scaffolds of the present study, greater collagen accumulation appeared in the implants with PEG scaffolds. However, it cannot be determined conclusively whether this is a direct result of the increased hydrophilicity, the faster degradation rate, or lower damping properties of the PEG scaffolds.

A biodegradable, elastomeric polyurethane scaffold that released basic fibroblast growth factor (bFGF) has been reported for soft tissue engineering applications. See, for example, Guan J., Stankus, J. J. and Wagner, E. R. Biodegradable elastomeric scaffolds with basic fibroblast growth factor release. J Control Release 120, 70, 2007.

Segmented PUR elastomers were synthesized from butane diisocyanate (BDI), putrescine, and poly(ε-caprolactone) diol. Scaffolds incorporating bFGF were processed using a thermally induced phase separation method. The scaffolds showed a two-stage release behavior characterized by an initial period of fast release (19-37% on day 1) followed by a second period of slow release over 4 weeks. The released bFGF was shown to induce proliferation of rat smooth muscle cells. However, in this study, the bFGF was released from a pre-formed polymer scaffold, not from a reactive polymer. PUR scaffolds prepared by reactive liquid molding of LDI, glycerol, water, and ascorbic acid (AA) have been shown to support controlled release of AA over 60 days. By dissolving the AA in the glycerol prior to adding the LDI, the AA was covalently bound to the polymer through reaction of the primary hydroxyl group in the AA with LDI to form urethane linkages. In the HDIt material of the present study, PDGF-BB was added as a lyophilized powder to minimize its reaction with the PUR. While the covalent binding approach was successful with a small molecule such as ascorbic acid, proteins were expected to lose their three-dimensional structure and denature upon reaction with the polymer. The faster release of PDGF-BB from the HDIt scaffolds compared to release of ascorbic acid from the LDI scaffolds is attributed to the absence of covalent bonds. As the scaffold swells with water, the PDGF dissolves and diffuses out of the scaffold, and the release is not dependent on the hydrolysis of covalent bonds.

Accordingly, biodegradable PUR scaffolds of the present invention prepared from triisocyanates using a one-shot process exhibited elastomeric mechanical properties and substantially lower compression set relative to scaffolds prepared from LDI. Their elastic behavior is thought to promote intimate contact between the material and surrounding tissue, which may facilitate ingrowth of new tissue and help keep the material in place when subjected to physiologically relevant strains. Both low- and high-damping elastomers can be synthesized by varying the glass transition temperature of the materials. Processing by two-component reactive liquid molding allows them to be injected and conform to the wound boundaries. The gel time of 3-5 minutes and moderate exotherm (e.g., <15° C. increase) suggests their utility for injectable wound healing applications. The materials supported cellular infiltration and generation of new tissue and facilitate neodermis formation with minimal inflammation. Signaling molecules were incorporated as labile powders upon synthesis, further enhancing their regenerative capabilities.

Further details and representative examples of the present invention are described in the following examples, which are presented to show embodiments of the present invention and are not to be construed as being limiting thereof.

### EXAMPLES

### Example 1

This Example demonstrates an aspect of the present invention, and more specifically a method of making a PUR scaffold of the present invention.

Glycolide and D,L-lactide were obtained from Polysciences (Warrington, Pa.), tertiary amine catalyst (TEGOAMIN33) from Goldschmidt (Hopewell, Va.), polyethylene glycol (PEG, MW 600 Da) from Alfa Aesar (Ward Hill, Mass.), and glucose from Acros Organics (Morris Plains, N.J.). Lysine triisocyanate (LTI) from Kyowa Hakko USA (New York), and hexamethylene diisocyanate trimer (HDIt, Desmodur N3300A) from Bayer Material Science (Pittsburgh, Pa.). PDGF-BB was a gift from Amgen (Thousand Oaks, Calif.). Sodium iodide (Na¹²⁵I) for radiolabeling was purchased from New England Nuclear (part of Perkin Elmer, Waltham, Mass.). Reagents for cell culture from HyClone (Logan, Utah). All other reagents were from Sigma-Aldrich (St. Louis, Mo.). Prior to use, glycerol and PEG were dried at 10 mm Hg for 3 hours at 80° C., and ε-caprolactone was dried over anhydrous magnesium sulfate, while all other materials were used as received.

Trifunctional polyester polyols of 900-Da and 1800-Da molecular weight (abbreviated as 900 and 1800) were prepared from a glycerol starter and 60% ε-caprolactone, 30% glycolide, and 10% D,L-lactide monomers, and stannous octoate catalyst, as published previously. These components were mixed in a 100-ml reaction flask with mechanical stirring under argon for 36 hours at 140° C. They were then dried under vacuum at 80° C. for 14 h.

PUR scaffolds were synthesized by one-shot reactive liquid molding of hexamethylene diisocyanate trimer (HDIt; Desmodur N3300A) or lysine triisocyanate (LTI) and hardener comprising either the 900-Da or 1800-Da polyol, 1.5 parts per hundred parts polyol (pphp) water, 4.5 pphp (1.5 pphp for LTI foams) TEGOAMIN33 tertiary amine catalyst, 1.5 pphp sulfated castor oil stabilizer, and 4.0 pphp calcium stearate pore opener. The isocyanate was added to the hardener and mixed for 15 seconds in a Hauschild SpeedMixer™ DAC 150 FVZ-K vortex mixer (FlackTek, Inc., Landrum, S.C.). This reactive liquid mixture then rose freely for 10-20 minutes. The targeted index (the ratio of NCO to OH equivalents times 100) was 115. To examine the effects of a hydrophilic polyether segment on the material properties, some materials were synthesized with poly(ethylene glycol) (PEG, 600 Da), such that the total polyol content consisted of 30 or 50 mol-% PEG and 70 or 50 mol-% of the polyester polyol.

Compression set of the scaffolds was determined using a TA Instruments Q800 Dynamic Mechanical Analyzer (DMA) in static compression mode (New Castle, Del.). After measuring their initial heights, triplicate 7 mm diameter cylindrical foam cores were compressed to 50% strain (i.e., 50% of their initial height) for 24 hours at room temperature according to ASTM standards. The samples recovered for 30 minutes, and then their final heights were measured. Compression set was calculated as the permanent deformation after the period of compressive stress, expressed as a percentage of the original height.

Core densities were determined from mass and volume measurements of triplicate cylindrical foam cores, of 7 mm diameter×10 mm height samples. The core porosities (ε_{C}) were subsequently calculated from the measured density values (ρ_{c}), where ρ_{P}=1200 kg m⁻³ is the polyurethane specific gravity and ρ_{A}=1.29 kg m⁻³ is the specific gravity of air.

$${ɛ}_{C} = 1 - ( \frac{{ρ}_{C}}{{ρ}_{P}} ) \frac{{ρ}_{P} - {ρ}_{A} {ρ}_{P} / {ρ}_{C}}{{ρ}_{P} - {ρ}_{A}}$$

The pore size and distribution were also assessed by scanning electron microscopy (Hitachi S-4200 SEM, Finchampstead, UK).

Temperature profiles of the reactive mixture during foaming were assessed with a digital thermocouple at the centers of the rising foams. Scaffold degradation rates in vitro were evaluated by measuring the mass loss at various time points up to 36 weeks of incubation of triplicate 10-mg samples in 1 ml phosphate buffered saline (PBS) (pH 7.4) at 37° C. as described previously. At each time point, the samples were rinsed in deionized water, dried under vacuum for 48 hours at room temperature, and weighed. The degradation media from 4 and 8 weeks were reserved for in vitro cell viability experiments.

### Example 2

This Example demonstrates thermal profile embodiments of the present invention. Thermal transitions of the materials were evaluated by differential scanning calorimetry (DSC) using a Thermal Analysis Q1000 Differential Scanning Calorimeter. 10-mg samples underwent two cycles of cooling (20° C./min) and heating (10° C./min), between −80° C. and 100° C.

DSC thermal profiles of the materials demonstrated single second-order glass transitions. The glass transition temperatures (Table 1, below), extrapolated from the steepest point of the heat flow (mW/mg) vs. temperature (° C.) curve during the second heating cycle, ranged from −30.7° C. (HDIt+50% PEG) to 6.4° C. (900/LTI). The glass transition temperature of the pure polyols, −41.7° C. (900-Da) and −44.7° C. (1800-Da), were significantly lower than those of the PUR networks. The substantial increase in the glass transition temperatures of the PUR networks relative to those of the pure polyols suggests that microphase-mixing of hard (isocyanate) and soft (polyol) segments has occurred. Addition of PEG proportionally depressed the glass transition temperatures. Use of the 1800-Da polyol also decreased the transition temperatures, perhaps due to enhanced microphase-separation of the larger soft segments. Glass transition temperatures were also measured by DMA using temperature sweeps (Table 1). Surprisingly, the values of T_{g} as measured by DMA were about 34-50° C. higher than those measured by DSC.

TABLE 1

| | Density | Porosity | Tg - DSC | Tg - DMA |
|---|---|---|---|---|
| Sample | kg m−3 | vol-% | (° C.) | (° C.) |
| 900/LTI | 87.5 ± 4.6 | 92.8 ± 0.4 | 6.4 | 56.6 |
| 1800/LTI | 86.2 ± 0.9 | 92.9 ± 0.1 | −16.2 | 23.8 |
| 900/HDIt | 98.2 ± 12.5 | 91.9 ± 1.0 | 0.2 | 40.3 |
| 1800/HDIt | 92.8 ± 7.7 | 92.4 ± 0.6 | −20.8 | 28.2 |
| HDIt + 30% PEG | 90.2 ± 2.6 | 92.6 ± 0.2 | −9.8 | 24.3 |
| HDIt + 50% PEG | 93.7 ± 11.4 | 92.3 ± 1.0 | −30.7 | 18.5 |

### Example 3

This Example demonstrates mechanical properties of embodiments of the present invention.

Dynamic mechanical properties were measured using the DMA in compression and tension modes. Cylindrical 7×6 mm samples were compressed along the axis of foam rise. The temperature-dependent storage modulus and glass transition temperature (T_{g}) of each material was evaluated with a temperature sweep of −80° C. to 100° C., at a compression frequency of 1 Hz, 20-μm amplitude, 0.3-% strain, and 0.2-N static force. The relaxation modulus was evaluated as a function of time with stress relaxation under 2-% strain and 0.2-N static force. The frequency-dependent storage modulus was also evaluated with a 0.1 to 10 Hz frequency sweep at a constant temperature of 37° C., with 0.3-% strain and 0.2-N static force. Stress-strain curves were generated by controlled-force compression of the cylindrical foam cores at 37° C. With an initial force of 0.1 N, each sample was deformed at 0.1 N/min until it reached 50% strain (i.e. 50% of its initial height). The Young's (elastic) modulus was determined from the slope of the initial linear region of each stress-strain curve (31). Due to their highly elastic properties, the scaffolds could not be compressed to failure. Therefore, as a measure of compressive strength, the compressive stress of triplicate cylindrical samples after one minute at 50% strain was measured using the DMA stress relaxation mode at 37° C. (29). Calculated from the measured force and cross-sectional sample area, the compressive stress indicates material compliance such that more compliant materials require lower stress to induce a particular strain.

Tensile testing was performed on thin, rectangular scaffold samples (10 mm long×5 mm wide×1.7 mm thick). Stress-strain curves were generated by elongating the samples at 1% strain per minute at 37° C. until failure. The Young's modulus was calculated as described above, and the tensile strength was determined as the stress (kPa) at failure.

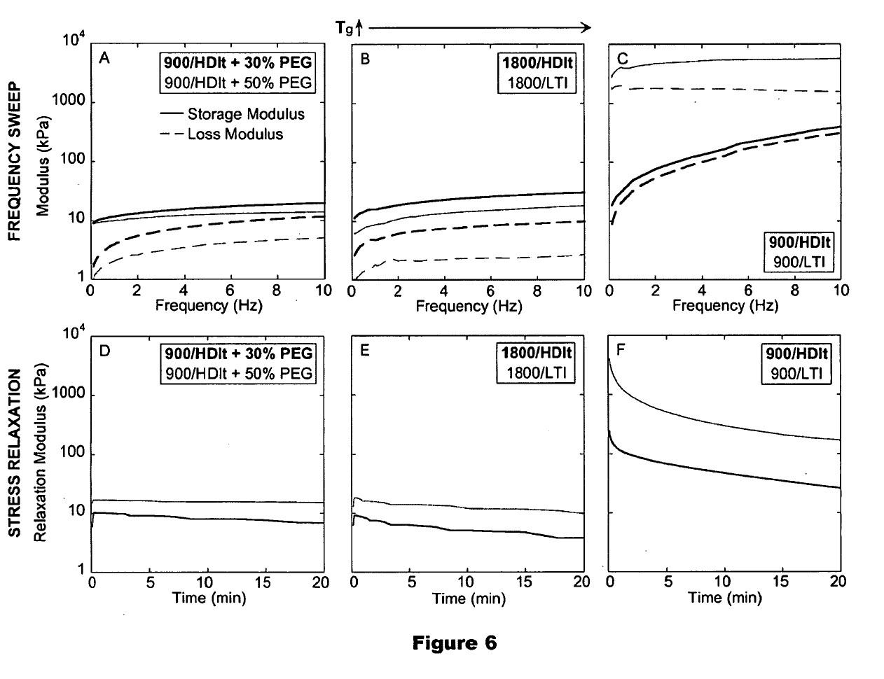

FIG. 6 shows the materials analyzed using stress relaxation and frequency sweep tests to evaluate their viscoelastic properties, which were shown to depend on the glass transition temperature. The six materials are organized into three groups in order of increasing temperature. The 900/HDIt+PEG materials (FIGS. 6a and d), which had DMA glass transition temperatures of 18.5° C. (50% PEG) and 24.3° C. (30% PEG), exhibited dynamic mechanical behavior similar to that of an ideal elastomer in the rubbery plateau zone. The storage modulus E′, which represents the energy stored elastically, was nearly constant over the entire frequency range (0.1-10 Hz), while the loss modulus E″, which represents the energy lost due to viscous dissipation, was very low at low frequencies and approaches E′ at higher frequencies (e.g., >5 Hz). Similarly, the stress relaxation data showed an initial increase in the relaxation modulus when the strain was applied, followed by a negligible (50% PEG) or slight (30% PEG) decrease in relaxation modulus over 20 minutes due to relaxation of the polymer network. Taken together, the frequency sweep and stress relaxation data suggest that the PUR scaffolds incorporating PEG are rubbery elastomers.

Frequency sweep and stress relaxation data are presented in FIGS. 6b and 6e for the 1800/LTI and 1800/HDIt materials, which have mechanical glass transition temperatures of 23.8° C. and 28.2° C., respectively. The 1800/HDIt material has a glass transition temperature closer to the experimental temperature (37° C.), and therefore exhibited viscoelastic properties representative of a material approaching the transition zone, where (a) the values of E′ and E″ increase with increasing frequency, and (b) the value of E″ approaches E′. As E″ approaches E′, an increasing fraction of the energy of deformation is dissipated as heat due to increased friction between polymer chains. The vibration damping properties of the material increase with increasing loss modulus E″. The frequency sweep data for the 1800/HDIt material show that E′ increased with increasing frequency and the value of E″ was close to that of E′, thereby suggesting that a substantial portion of the energy of deformation was dissipated as heat. The stress relaxation data are in qualitative agreement with the frequency sweep data. The relaxation modulus increased to about 10 kPa when the strain was applied, and then decreased over 20 minutes. At short times (corresponding to high frequencies), the period is too short to enable an active segment of the network to exhibit all possible conformations. Therefore, the strain resulting from a given stress is less than that at longer times (lower frequencies); thus the relaxation modulus is expected to decrease with increasing time (decreasing frequency).

In FIGS. 6c and 6f, the frequency sweep and stress relaxation data are presented for the 900/LTI (T_{g}=56.6° C.) and 900/HDIt (T_{g}=40.3° C.) materials. The 900/HDIt material has a T_{g} slightly greater than 37° C., and therefore exhibited properties typical of the transition zone. The moduli E′ and E″ increased with increasing frequency, and the values of E″ were close to E′. In the stress relaxation experiments, the relaxation modulus initially reached a high value when the strain was applied and then decayed over 20 minutes by an order of magnitude. The 900/LTI material has a T_{g} substantially greater than 37° C., and therefore exhibited properties typical of the glassy zone, characterized by storage modulus 2-3 orders of magnitude greater than that in the rubbery plateau. Furthermore, the values of E′ and E″ did not change substantially with increasing frequency.

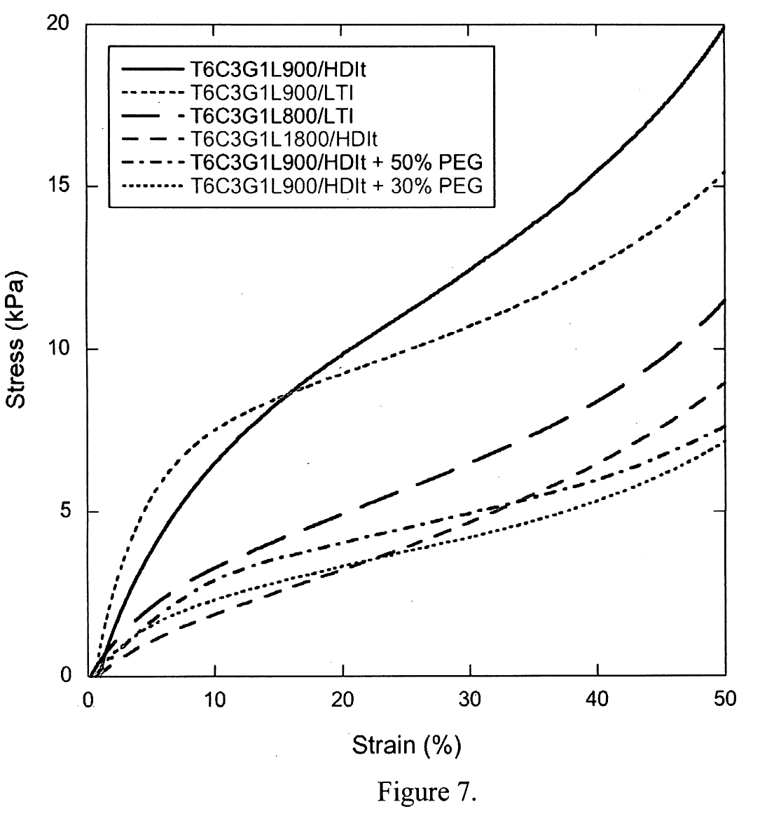

Stress-strain plots show elastomeric behavior of the PUR scaffolds even up to 50% compressive strain (FIG. 7). The Young's moduli, calculated from the slope of the initial linear region of the stress-strain curves, are listed in Table 2. 900/LTI scaffolds exhibited the highest modulus values, followed by the 900/HDIt materials, while the 1800-Da polyol or additional PEG appeared to reduce the modulus of the scaffolds. The modulus differences among the materials were statistically significant (p<0.005). The compressive stress at 50% strain ranged from 4.8 to 10.5 kPa for the different scaffold formulations (Table 2), and the addition of PEG reduced the compressive stress relative to the equivalent scaffold without PEG. The two materials with PEG had nearly equivalent compressive stress values, but all other differences were statistically significant (p<0.005).

The tensile strength and Young's modulus of the thin scaffold samples are given in Table 2, below. They were both determined from stress-strain curves performed until sample failure. The trend is similar to the compressive strengths, where the 900/LTI materials had the highest tensile strength (266.5±33.6 kPa), followed by the 900/HDIt materials (33.6±9.1 kPa). Use of the 1800-Da polyol or PEG decreased the modulus and strength. The Young's moduli of 1800/LTI, 1800/HDIt, and 900/HDIt+30% PEG were statistically similar (0>0.05), but all other tensile strength differences were statistically significant (p<0.005).

TABLE 2

| | Young's | Young's | | |
|---|---|---|---|---|
| | Modulus | Modulus | Tensile | |
| | Compression | Tension | Strength | Strain at |
| Sample | (kPa) | (kPa) | (kPa) | break (%) |
| 900/HDIt | 50.5 | 38.8 ± 8.0 | 33.6 ± 9.1 | 103.9 ± 34.5 |
| 900/LTI | 201.8 | 121.8 ± 43.3 | 266.5 ± 33.6 | 216.3 ± 75.2 |
| HDIt + 30% | 46.2 | | | |
| PEG | | | | |
| HDIt + 50% | 42.4 | 43.9 ± 17.0 | 20.1 ± 5.0 | 59.2 ± 23.0 |
| PEG | | | | |

### Example 4

This Example demonstrates in vitro biocompatibility of embodiments of the present invention.

MC3T3-E1 embryonic mouse osteoblast precursor cells were statically seeded onto thin foam discs (25×1 mm) at 5×10⁴ cells per well in 24-well tissue-culture polystyrene plates. The cells were cultured with 1 ml α-minimum essential medium (α-MEM) per well, containing 10% fetal bovine serum, 1% penicillin (100 units/ml) and streptomycin (100 μg/ml). After 5 days, the cell-seeded scaffolds were removed from culture, washed with PBS, and transferred to a new 24-well plate to verify cell adherence to the materials. 4 μM Calcein AM from the Invitrogen-Molecular Probes Live/Dead Viability/Cytotoxicity Kit for mammalian cells (Eugene, Oreg.) was added to the samples. Calcein AM dye is retained within live cells, imparting green fluorescence (excitation/emission: 495/515 nm). Cell viability was assessed qualitatively by fluorescent images acquired with an Olympus DP71 camera attached to a fluorescent microscope (Olympus CKX41, U-RFLT50, Center Valley, Pa.).

In addition, PUR degradation products from 4 and 8 weeks were analyzed for cell viability and cytotoxicity. The same MC3T3-E1 cells were seeded at 5×10³ cells per well in a 96-well plate with 90 μl cell culture medium (described above) and 10 μl degradation media or PBS control. After the cells were cultured for 72 hours, the media was removed, the wells were rinsed with fresh PBS, and 2 μM Calcein AM was added to the wells. The percentage of viable cells was assessed by quantifying the fluorescence of the samples, in comparison to wells that were cultured with all 100 μl cell culture media, with a Biotek fluorescence microplate reader (Winooski, Vt.).

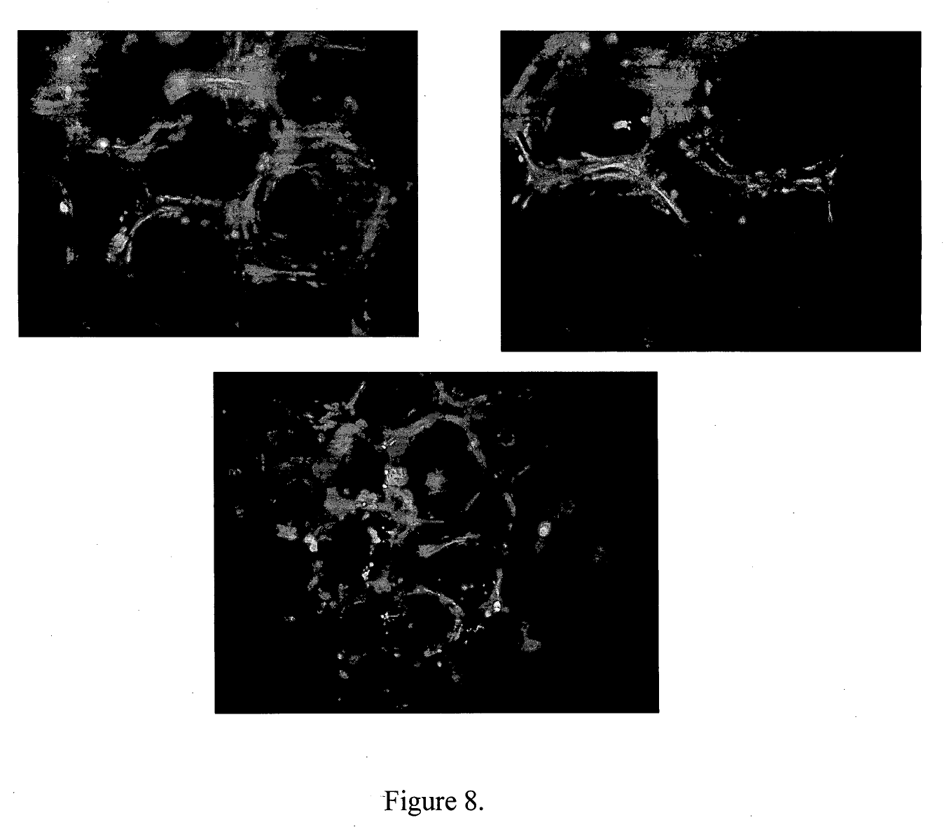

The MC3T3 cells permeated and adhered to the scaffold interstices, as shown by fluorescent microscope images (FIG. 8). Live cells, as indicated by dye uptake, remained attached to the scaffold during transfer procedures. The cells were easily discriminated from the autofluorescent scaffold material.

The percent viability (Table 3, below) was determined as the proportion of live cells, or fluorescence intensity, in the wells cultured with the 4-week and 8-week degradation products, in comparison to that of cells cultured in media only. Cells cultured with 10 μl PBS exhibited 94.7% viability, while the 4-week and 8-week degradation samples yielded 87.5-94.7% and 88.4-89.9% viability, respectively. All differences, including the PBS control sample, were not statistically significant (p>0.5).

TABLE 3

| | | | HDIt + 50% | |
|---|---|---|---|---|
| | LTI | HDIt | PEG | Control (PBS) |
| 4 weeks | 93.7 ± 8.2% | 94.7 ± 8.6% | 87.5 ± 11.6% | 94.7 ± 10.9% |
| 8 weeks | 89.1 ± 8.0% | 88.4 ± 5.5% | 89.9 ± 10.1% | |

### Example 5

This Example demonstrates in vivo biocompatibility of embodiments of the present invention.

After polymerization, the materials were cut into 8×2 mm discs for in vivo implantation to assess biocompatibility and degradation properties. The discs were sterilized for 5 minutes in ethanol prior to dorsal subcutaneous implantation in adult male Sprague-Dawley rats. Implants were retrieved from euthanized animals at 5, 14, and 21 days post-implantation, fixed in formalin for 24 hours, embedded in paraffin, and processed for histological evaluation with Gomori's trichrome as well as hematoxylin and eosin staining.

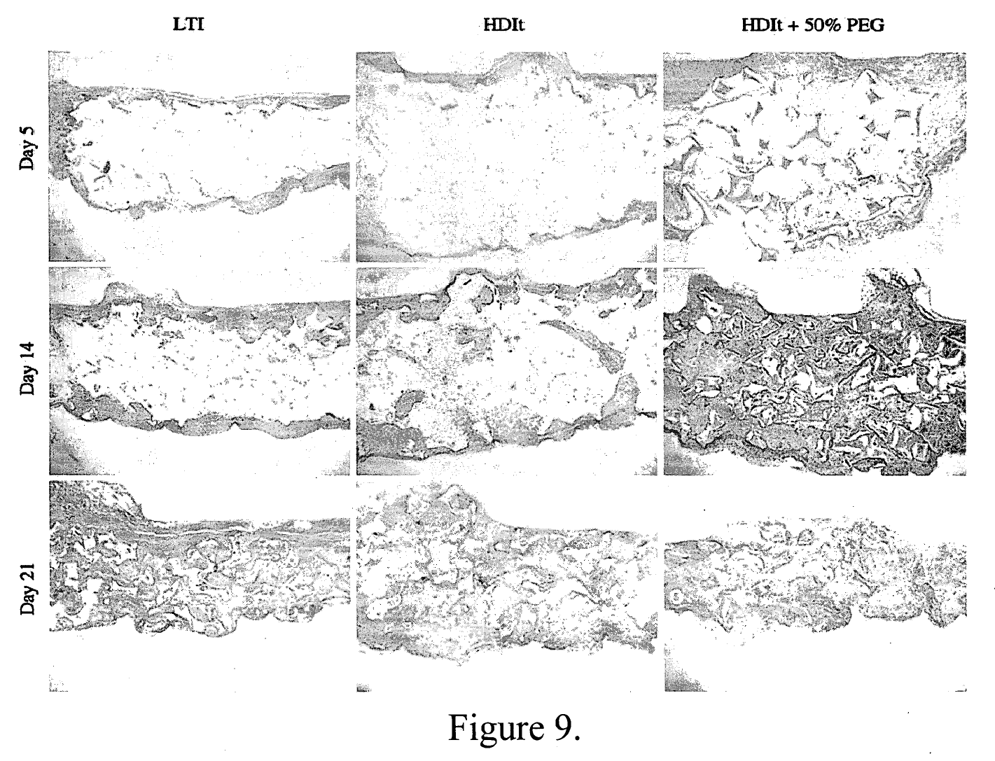

Tissue response was evaluated by subcutaneous implantation of 2×8 mm discs of each formulation in rats for up to 21 days (FIG. 9). During this time, initial infiltration of plasma progressed to the formation of dense granulation tissue. Hence, the implant served as a model of deep wound healing. All of the implants showed progressive invasion of granulation tissue with little evidence of an overt inflammatory response or cytotoxicity. Fibroplasia and angiogenesis appeared to be equivalent among the different formulations. Extracellular matrix with dense collagen fibers progressively replaced the characteristic, early cellular response. The LTI scaffolds exhibited a greater extent of degradation at 21 days, although the incorporation of PEG into the HDIt scaffold accelerated its degradation significantly. Degradation rates were much higher in vivo. With time, each of the materials showed signs of fragmentation and engulfment by a transient, giant cell, foreign body response. After the remnant material was resorbed, giant cells were no longer evident.

### Example 6

This Example demonstrates embodiments of the present invention including the incorporation of radiolabeled PDGF-BB.

PDGF-BB was labeled with radioactive iodine (¹²⁵I) using IODO-BEADS Iodination Reagent (Pierce Biotechnology, Rockford, Ill.). The IODO-beads were incubated in 1 ml Reaction Buffer containing sodium iodide (approximately 1 mCi per 100 μg of protein) for 5 minutes at room temperature. 110 μl PDGF solution (0.43 mg/ml in PBS) was added to the IODO-BEADS reaction solution and incubated for 25 minutes at room temperature. The solution was then removed from the IODO-BEADS reaction tube and the ¹²⁵I-labeled PDGF (¹²⁵I-PDGF) was separated in a Sephadex disposable PD-10 desalting column (Sigma-Aldrich). Eluted fractions of 200 μl each were collected and analyzed by a Cobra II Autogamma counter (Packard Instrument Co, Meridien, Conn.) to identify the fractions containing the ¹²⁵I-PDGF.

The ¹²⁵I-PDGF was then co-dissolved and lyophilized with heparin and glucose in order to stabilize the protein during lyophilization and scaffold synthesis. Final dosages were 10 μg and 50 μg ¹²⁵I-PDGF per gram of foam, each with 0.5 mg heparin and 20 mg glucose per gram of foam. The lyophilized powder was added to the polyol hardener component, which included 50 mol-% PEG, before mixing with the isocyanate to prepare the PUR scaffolds.

The initial ¹²⁵I-PDGF levels in triplicate 50-mg samples for each ¹²⁵I-PDGF dosage were first measured by the Autogamma counter and then incubated in 1 ml MEM non-essential amino acid solution containing 1% BSA contained in glass vials while mixed end-over-end at 37° C. MEM and BSA were included to mimic the cellular growth environment and minimize adsorption of PDGF onto the scaffolds and vials. The buffer was removed and refreshed from each vial every day for the first 4 days, and then every two days until 28 days. The ¹²⁵I-PDGF concentrations in the release samples were quantified by the Autogamma counter.

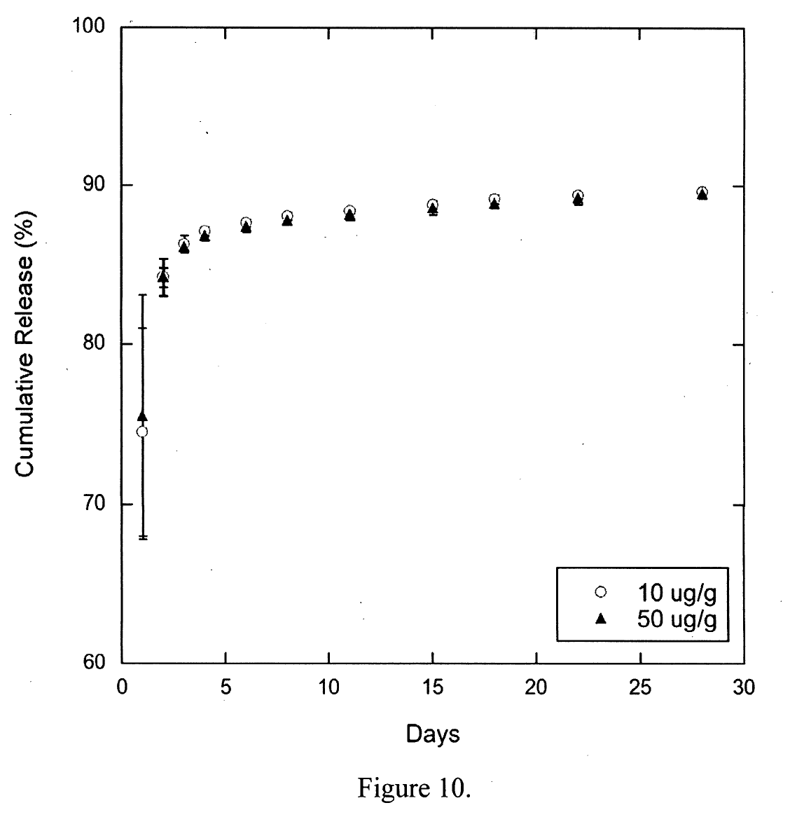

The in vitro release of ¹²⁵I-PDGF from the polyurethane scaffolds, for both 10 μg and 50 μg ¹²⁵I-PDGF per gram of foam, is shown in FIG. 10. The release profiles are essentially identical for each of the two dosages. The cumulative % release is defined as the cumulative elution of ¹²⁵I-PDGF at each time point divided by the total ¹²⁵I-PDGF in each sample. The scaffolds showed a two-stage release profile, characterized by a 75% burst release within the first 24 hours, and slower release thereafter. By 21 days, 89% of the ¹²⁵I-PDGF had eluted from the scaffolds.

### Example 7

This Example demonstrates an additional aspect of the present invention related to PEUUR foam synthesis.

Materials & Methods

Materials. Sulfated castor oil (turkey red oil), calcium stearate, stannous octoate, glycerol, and ε-caprolactone were purchased from Aldrich (St. Louis, Mo.). Glycolide and D,L-lactide were obtained from Polysciences (Warrington, Pa.), tertiary amine catalyst (TEGOAMIN33) from Goldschmidt (Hopewell, Va.), and polyethylene glycol MW 600 (PEG 600) from Alfa Aessar (Ward Hill, Mass.). Lysine triisocyanate (LTI) was received from Kyowa Hakko USA (New York), and hexamyethylene diisocyanate trimer (HDIt, Desmodur N3300A) was purchased from Bayer MaterialScience (Pittsburgh, Pa.). Prior to use, glycerol and PEG 600 were dried at 10 mm Hg for 3 hours at 80° C., and ε-caprolactone was dried over anhydrous magnesium sulfate.

Polyester polyol synthesis. 900-Da and 1800-Da polyester triols (P6C3G1L900, P6C3G1L1800, P723G1L900) were prepared from a glycerol starter and the appropriate ratios of caprolactone/glycolide/lactide monomers (60/30/10 and 70/20/10), and stannous octoate catalyst (Aldrich). These components were mixed in a 100-mL reaction flask with mechanical stirring under argon atmosphere for 36 hours at 140° C. The completed polyols were then dried, unwashed, under vacuum at 80° C. for 14 hours. The polyester polyols were used without precipitation or washing, as there washing does not seem to significantly affect the polyol hydroxyl number. The particular ratios of ε-caprolactone, glycolide, and D,L-lactide monomers used for these polyols were chosen to evaluate the effects of their contrasting half-lives of 20 days (6C3G1L) and 225 days (7C2G1L).

Polyesterpolyol characterization. The polyester triol molecular weights were assessed by gel permeation chromatography (GPC) with two Mesopore columns (Polymer Laboratories, Amherst, Mass.) and a Waters 2414 Refractive Index Detector (Milford, Mass.). The triols were dissolved to 0.5% in tetrahydrafuran, run through the columns at 1 mL/min, and evaluated relative to low-MW polystyrene standards. The polyols were dissolved in dichloromethane and analyzed by solution-phase nuclear magnetic resonance (NMR), using a Bruker 300 MHz NMR (Billerica, Mass.), to verify the extent of reaction and chemical structure of the polyols.

The hydroxyl numbers of the triols were measured according to an ASTM NCO titration method, because the corresponding OH titration method was inaccurate due to side reactions. The OH numbers, calculated from the number-average molecular weight (Mₙ) and functionality (f) of the triols, determine the formulation of the PEUUR foams assuming complete conversion of the triol monomers.

$$OH No . = \frac{56.1 \times {10}^{3} f}{{M}_{n}} \qquad ( 1 )$$

HDI monomer was added to each polyol at a 4:1 NCO:OH equivalent ratio to produce a prepolymer, using dibutyl dilaurate as a catalyst. The components were combined, in a 50-mL reaction flask and heated to 70° C. under argon for 3 hours. The prepolymer was subsequently dissolved in warm toluene, reacted with excess dibutylamine, and the reaction stopped with methanol. This excess dibutylamine was determined by back titration with standardized 1 M HCl using a Metrohn Titrino. The polyol % NCO was calculated from the following formula, where V represents the volumes of HCl added for titration of the blank and sample, C_{HCl} is the concentration of HCl, and Wₛₐₘₚₗₑ is the mass of polyol reacted with dibutylamine.

$$\% NCO = \frac{( {V}_{mean blank} - {V}_{sample} ) \times {C}_{HCl} \times 42.01}{{W}_{sample}} \qquad ( 2 )$$

The OH Number was then computed from the % NCO with the following equation, where M_{HDI} and M_{polyol} are the masses of each combined to make the prepolymer, and % NCO_{HDI} is specified by the manufacturer.

$$OH No . = - [ \frac{( \% NCO \times ( {M}_{HDl} + {M}_{polyol} ) ) - ( {M}_{HDl} \times \% {NCO}_{HDl} )}{42.01} ] \times ( \frac{56.1 \times 1000}{{M}_{polyol}} )$$

PEUUR foam synthesis. The PEUUR foams were synthesized by reactive liquid molding of a hardener and isocyanate. The hardener contained the polyester triol, 1.5 parts per hundred parts polyol (pphp) water, 4.5 pphp TEGOAMIN33 tertiary amine catalyst, 1.3 or 1.5 pphp sulfated castor oil (stabilizer), and 4.0 pphp calcium stearate (pore opener). For foams that contained PEG, the one hundred parts of polyol were divided between the polyester triol and PEG at ratios of 70/30 and 50/50. In embodiments, the PEG is present in an amount less that about 60%. The isocyanate component consisted of 111.1 pphp HDIt or 52.0 pphp LTI. Once the isocyanate was added to hardener in a small plastic cup, the mixture was mixed in a Hauschild SpeedMixer™ DAC 150 FVZ-K vortex mixer (FlackTek, Inc., Landrum, S.C.) for 15 seconds, and then allowed to rise freely, about 10-20 minutes. The NCO groups of the isocyanate react with the water to form carbon dioxide, which acts as a “blowing agent” to foam the mixture.

Density & porosity. The PEUUR foam core densities were determined from mass and volume measurements of triplicate cylindrical foam cores, cut with a cork borer for approximately 7×10 mm (diameter×height) samples. The core porosities (ε_{C}) were subsequently calculated from these density values, where ρ_{P}=1200 kg m⁻³ is the polyurethane specific gravity and ρ_{A}=1.29 kg m⁻³ is the specific gravity of air.

$${ɛ}_{C} = 1 - ( \frac{{ρ}_{C}}{{ρ}_{P}} ) \frac{{ρ}_{P} - {ρ}_{A} {ρ}_{P} / {ρ}_{C}}{{ρ}_{P} - {ρ}_{A}}$$

The pore morphologies—pore size and distribution—were also assessed by scanning electron microscopy (SEM) with a Hitachi S-4200 Scanning Electron Microscope.

Infrared analysis. The chemical composition of the foams was evaluated by Fourier transform infrared spectroscopy (FT-IR) using a Bruker Tensor 27 FT-IR (Billerica, Mass.). The foams were sliced thinly and analyzed directly under transmittance mode.

Degradation. The in vitro degradation rates of the foams were evaluated by measuring the mass loss after 1, 2, 4, 8, and 12 weeks of incubation of triplicate 10-mg samples in 1 mL PBS at 37° C. At each time point, the samples were rinsed in diH₂O, dried under vacuum for 48 hours at 37° C., and weighed to measure their mass loss with time.

Thermal analysis. The thermal decomposition profiles of the foams were ascertained by thermal gravimetric analysis (TGA). Samples of 3 to 6 mg were heated from 25° C. to 600° C. at 20° C./min in an Instrument Specialist TGA 1000. The thermal glass transition temperatures (T_{g}) were then evaluated by differential scanning calorimetry (DSC) on a Thermal Analysis Q1000 Differential Scanning Calorimeter. 10-mg samples underwent two cycles of cooling to −80° C. at 20° C./min with nitrogen gas and heating to 100° C. at 10° C./min.

Dynamic mechanical analysis. The foam mechanical properties were assessed by dynamic mechanical analysis (DMA) in compression mode. Cylindrical 7×6 mm cores were compressed along the same axis in which the foam rose during synthesis. The temperature-dependent storage modulus and mechanical glass transition temperature of each foam was evaluated with a temperature sweep of −80° C. to 100° C., at a compression frequency of 1 Hz, 20-μm amplitude, 0.3-% strain, and 0.2-N static force. The frequency-dependent storage modulus was also evaluated with a 0.1 to 10 Hz frequency sweep at a constant temperature of 37° C., 0.3-% strain, and 0.2-N static force. The foam relaxation modulus was evaluated as a function of time with stress relaxation under 2-% strain and 0.2-N static force.

Compression testing. Stress-strain curves were generated by controlled-force compression of the cylindrical foam cores at 37° C. With an initial force of 0.1 N, each sample was deformed at 0.1 N/min until it reached 50% strain (i.e. 50% of its initial height). The Young's (elastic) modulus was determined from the slope of the initial linear region of each stress-strain curve. The compressive stress of triplicate 7×9 mm foam cores after one minute at 50% strain was measured using the DMA stress relaxation mode at 37° C. Calculated from the measured force and cross-sectional area of foam sample, the compressive stress indicates material compliance such that more compliant materials require lower stress.

In vivo analysis. Four of the foam formulations (6C3G1L900/HDIt, 6C3G1L900/50PEG/HDIt, 6C3G1L900/LTI, 7C2G1L900/LTI) were cut into 8×2 mm discs for in vivo implantation to assess biocompatibility and degradation properties. The discs were implanted into full-thickness excisional dorsal wounds in adult Sprague-Dawley rats. The wounds were splinted with stainless steel washers for 7 days to prevent wound contraction and thereby allow the normal wound filling and granulation tissue infiltration typical in humans. Semi-occlusive dressing held the foam discs in place and protected the wound. The discs were also implanted subcutaneously in the rats to evaluate biocompatibility. Wounds were harvested at days 5, 14, and 21 and processed for Gomori's trichrome histological evaluation.

Results

Polyester polyol characterization. The polyol number-average and weight-average molecular weights, as determined by GPC, are given in Table 4, below. These molecular weights are consistently greater than the target values of 900 and 1800 g/mol, most likely because they are measured relative to the GPC weight standards, rather than as absolute values. This trend has been reported similarly in previous reports. The NMR spectra of each of the polyols showed that synthesis had proceeded to completion, with no detectible peaks representing free monomer.

Table 4 provides the polyol % NCO and OH Numbers, as measured by NCO titration, which were used to determine the foam compositions and index numbers. These measured OH Numbers are within 10% (900-MW polyols) and 30% (1800-MW polyol) of the theoretical OH Numbers, which were calculated based on the polyol compositions.

TABLE 4

| | Mₙ | M_{w} | | | Theoretical | Actual | T_{g} |
|---|---|---|---|---|---|---|---|
| Polyol | (g/mol) | (g/mol) | PDI | % NCO | OH # | OH # | (° C.) |
| T6C3G1L900 | 1422 | 2031 | 1.43 | 12.26 | 186.78 | 210.44 | −41.66 |
| T6C3G1L1800 | 3176 | 4105 | 1.29 | 11.81 | 94.41 | 125.35 | −44.73 |
| T7C2G1L900 | 1432 | 2086 | 1.46 | 12.57 | 187.14 | 202.47 | −38.22 |

PEUUR Foam Characterization

Density & porosity. The average foam densities ranged from 84.9'14.0 to 98.2±7.5 kg m⁻³, with porosities from 91.9±1.0 to 93.0±1.2 vol-% (Table 5, below). Foams made from a given isocyanate seemed to have lower density—and therefore higher porosity—when made with 1800-MW polyol than with 900-MW polyol. Likewise, LTI foams tended to have lower densities than HDIt foams. SEM images illustrated the pores to be almost uniformly spherical, 200-400 μm in diameter, and highly interconnected. In other words, numerous openings in the foam walls connect the individual pores. Addition of PEG had negligible effect on the foam density and porosity, but SEM shows that the pores were more irregularly shaped and variable in size, reaching 500 μm in diameter.

TABLE 5
PEUUR foam properties.

| | | Density | Porosity | DSC | DMA | Storage Mod. | Young's Mod. |
|---|---|---|---|---|---|---|---|
| Polyol | Isocyanate | (kg/m³) | (vol-%) | T_{g} (° C.) | T_{g} (° C.) | (37° C., MPa) | (37° C., kPa) |
| 6C3G1L900 | HDIt | 98.2 ± 12.5 | 91.9 ± 1.0 | 0.2 | 40.3 | 0.723 | 0.505 |
| 6C31LG1800 | HDIt | 92.8 ± 7.7 | 92.4 ± 0.6 | −20.8 | 28.2 | 0.037 | 0.260 |
| 6C3G1L900 | LTI | 87.5 ± 4.6 | 92.8 ± 0.4 | 6.4 | 56.6 | 10.918 | 2.019 |
| 6C31LG1800 | LTI | 86.2 ± 0.9 | 92.9 ± 0.1 | −16.2 | 23.8 | 0.111 | 0.564 |
| 7C2G1L900 | LTI | 84.9 ± 14.0 | 93.0 ± 1.2 | −5.0 | 37.6 | 0.853 | 0.856 |
| 6C3G1L900/ | HDIt | 90.2 ± 2.6 | 92.6 ± 0.2 | −9.8 | 24.3 | 0.014 | 0.462 |
| 30PEG | | | | | | | |
| 6C3G1L900/ | HDIt | 93.7 ± 11.4 | 92.3 ± 1.0 | −30.7 | 18.5 | 0.018 | 0.424 |
| 50PEG | | | | | | | |

Infrared analysis. FT-IR analysis produces characteristic vibration peaks for the ester (1765, 1303, & 1114 cm⁻¹), urethane (3422 & 1765 cm⁻¹), and urea (1469 cm⁻¹) groups (data not shown). There is no evident NCO peak at 2285-2250 cm⁻¹, which implies that most of the free NCO has reacted upon foaming. The absence of a peak near 1710 cm⁻¹ suggests that there is negligible hydrogen bonding of the urethane groups.

Degradation. The PEUUR foams seem to degrade more quickly in vivo than in vitro.

Thermal analysis. Upon heating, TGA shows that the foams begin to decompose at 200° C., while only 10% of the material remains at 500° C. and 0% at 600° C. (data not shown). The foams all have similar decomposition profiles, besides slightly faster mass loss for LTI foams from 350 to 500° C. DSC thermal profiles of the pure polyols and foams demonstrated single, second-order glass transitions, where the heat flow required to maintain sample temperature increases with an endothermic glass transition. The glass transition temperatures (T_{g}), extrapolated from the steepest point of the output curve of heat flow (mW/mg) vs. temperature (° C.), ranged from −30.7° C. for the HDIt foam with 50% PEG to 6.4° C. for the T6C3G1L900/LTI foam (Table 5). The T_{g}'s of the pure polyols, at −44.7° C. to −38.22° C., are significantly lower than those of the foams (Table 4). The presence of only one thermal transition and the distinct difference between the T_{g}'s of the pure polyol and foam suggests that phase-mixing of hard (isocyanate) and soft (polyol) segments has occurred within the foam. Increasing the polyol molecular weight from 900 to 1800 g/mol caused the T_{g} to decrease for both the HDIt and LTI foams, perhaps due to larger soft-segment blocks. Addition of PEG likewise depresses the foam glass transition temperatures.

Dynamic mechanical analysis. Mechanical T_{g}'s were identified as the temperature at the maximum tan δ in a DMA temperature sweep, where tan δ is the derivative of the storage modulus. The T_{g}'s ranged from 18.5° C. to 56.6° C., with an apparent trend of lowered T_{g} with increased PEG content or polyol molecular weight (Table 5). While the trends followed those of the thermal T_{g}'s determined by DSC, the mechanical T_{g}'s were consistently higher by approximately 40° C. The storage modulus of each foam at 37° C. depended on whether the T_{g} fell above or below 37° C. For example, T6C3G1L900/LTI, with a T_{g} of 56.6° C., is somewhat glassy at 37° C. and has a high storage modulus. T7C2G1L900/LTI and T6C3G1L900/HDIt are undergoing glass transition at 37° C. with storage moduli near 0.1 MPa, while the others are in the rubbery plateau region with even lower storage moduli.

Frequency sweeps of the foams at 37° C. resulted in similar trends as for the temperature sweeps. Foams in the glassy regime at 37° C. demonstrated the highest storage moduli, which decreased as foams tended toward the glass transition and rubbery plateau. The storage modulus increased by approximately 10% for all foams as the frequency was raised from 0.1 to 10 Hz.

Stress relaxation experiments illustrated the relative elasticity or plasticity of the foams at 37° C., depending on the slope of the curve over the duration of relaxation time. Foams with either the 1800-MW polyol or additional PEG demonstrated higher elasticity, as they maintained a relatively constant relaxation modulus over time with applied strain. Once again, the relative magnitudes of the relaxation moduli mirrored the trends of the storage moduli from the temperature and frequency sweeps.

Compression testing. Stress-strain plots represent typical elastomeric behavior. The Young's moduli, calculated from the slope of the initial linear region of the stress-strain curves, spanned from 0.3 to 2.0 kPa (Table 5). The compressive stress at 50% strain ranged from 5 to 15 kPa for the different foam formulations. HDIt and LTI foams made with the same polyol produced similar compressive stress values, and the foams with 1800-MW polyol demonstrated lower stress than the foams with 900-MW polyol. The addition of PEG caused a decrease in the compressive stress from that of the equivalent foams without PEG, although the values were nevertheless higher than the foams with 1800-MW polyol.

In vivo analysis. The scaffolds had significant permeation of new granulation tissue and some material degradation by day 14. Degradation was more extensive by day 21, along with the presence of mature granulation tissue, dense collagen fibers, and a giant cell response associated with only the material remnants. The excisional wounds exhibited almost complete epithelization and limited fibrous encapsulation by day 21. Infiltration of granulation tissue occurred from the basolateral surfaces of the implant. The LTI foams exhibited greater degradation than the HDIt foam at day 21, although addition of PEG to the HDIt foam seemed to accelerate its degradation significantly, such that it nearly approximated that of the LTI foams.

Without any supplementary fillers, foams synthesized from trifunctional isocyanates seem to be more resilient than those from difunctional isocyanates. This can be observed in a direct comparison of compression set results of LDI and LTI foams made with the same polyol. Structural rigidity and resiliency of the foams most likely depends on the frequency of urethane linkages, because FT-IR spectra show no evidence of physical crosslinking, such as hydrogen bonding, in foams from either di- or triisocyanates. Thus it can be deduced that the higher functionality of triisocyanate provides a greater extent of chemical crosslinking between the polyol and isocyanate phases. The sulfated castor oil stabilizer is added during foam synthesis to encourage miscibility of the two phases and therefore the incidence of urethane linkage formation.

Materials in wound healing applications could benefit from greater resiliency, which would allow them to better conform to the wound site and maintain their shape upon compression or movement.

The thermal properties of the foams depend more on the polyol composition than on the given triisocyanates. For example, the using LTI instead of HDIt for a T6C3G1L900 foam causes the glass transition temperature to increase only slightly, from 0.2 to 6.4° C. On the other hand, using T6C3G1L1800 (T_{g} 44.7° C.) instead of T6C3G1L900 (T_{g} 41.7° C.) results in a significant decrease in T_{g}, from 0.2 to −20.8° C. for the HDIt foam, and 6.4 to −16.2° C. for the LTI foam. The presence of only a single thermal transition, the glass transition, in the DSC thermograms suggests that the hard and soft segments are well integrated and not micro-phase separated. Furthermore, the glass transition temperature of each foam differs significantly from that of its constituent polyol. The placement of the foam T_{g}'s below 37° C., implies that the soft polyol segments are amorphous at this temperature, which may contribute to their greater influence on the foam thermal properties. An increase in polyol molecular weight may cause the individual soft segments to lengthen, although the overall soft-segment content remains constant. Since this polyol soft segment has a lower T_{g}, it stands to reason that longer polyol segments cause the foam T_{g} to decrease substantially.

Previous studies showed that in vitro degradation, which is almost entirely hydrolytically driven, depended greatly on the polyol composition. However, all the materials degraded much faster in vivo than in vitro, an observation that has been documented previously for porous poly(D-lactic-co-glycolic acid) foams. Furthermore, the relative degradation rates do not translate from in vitro to in vivo environments. This is most likely because any enzymatic degradation and material elimination by the giant cell response overrides the rate of hydrolytic degradation in vivo.

Lysine triisocyanate is a favorable component of polyurethane scaffolds, because it displays the suitable biological properties of a lysine-based isocyanate with the enhanced mechanical properties of a triisocyanate. However, because of the limited commercial availability of LTI, we must examine the viability of other triisocyanates. Hexamethylene diisocyanate trimer foams display similar thermal and mechanical characteristics to LTI foams, except with slower in vivo degradation. This discrepancy, however, was overcome when PEG 600 was added to the HDIt foams.

The foam samples that behave most elastically in the DMA stress relaxation experiments also have the lowest T_{g}'s—the two T6C3G1L1800 foams and both T6C3G1L900/PEG600/HDIt foams. This is not a coincidence, since the lower T_{g} allows them to be in the rubbery plateau region at 37° C., as shown by the DMA temperature sweep experiments. Even a slight decrease in the mechanical glass transition temperature, from above to below 37° C., dramatically changes the mechanical strength of the foams. A 16-° C. drop in T_{g}, from 56.6° C. (T6C3G1L900/LTI) to 40.3° C. (T6C3G1L900/HDIt) causes an order of magnitude reduction in the storage modulus, from 10.9 to 0.7 MPa. A subsequent 12-° C. drop in T_{g} results in another order of magnitude decrease in the storage modulus, to 0.04 MPa (T6C3G1L1800/HDIt, T_{g} 28.2° C.). The capability to change the mechanical properties of the foams so greatly with relatively small changes in T_{g} allows for versatility and a wide range of material properties. Moreover, we have shown that we can control the glass transition temperatures at the molecular-level by altering the polyol composition and molecular weight, as well as by adding other components such as PEG 600.

The 600-MW poly(ethylene glycol) acts as a plasticizer when added to the foams, as it causes the thermal glass transition temperature of the T6C3G1L900/HDIt foam to drop from 0.2 to −9.8° C. for 30% PEG and −30.7° C. for 50% PEG. While this is a large temperature drop, it does not significantly affect the structural properties of the foams since these temperatures are all below our mean operating temperature of 37° C. More importantly, PEG causes the mechanical T_{g} of the T6C3G1L900/HDIt foam to drop from 40.3 to 24.3° C. for 30% PEG and 18.5° C. for 50% PEG. This decrease in T_{g} from above to below 37° C. is particularly significant because it changes the phase of the material at body temperature from glassy to rubbery. As the glass transition typically accompanies at least a two orders of magnitude reduction in storage modulus, we observe huge effects on the mechanical strength of the materials when PEG is incorporated into the foams. PEG also has a significant effect on the in vivo behavior of the foams. After 21 days in the excisional wound, nearly twice as much of the T6C3G1L900/HDIt foam with 50% PEG had degraded as that without PEG. This is perhaps because its hydrophilic nature encourages more cellular interaction, and therefore accelerated cell-mediated degradation.

These polyurethane foams demonstrate the necessary qualities to be a successful injectable biomaterial for wound healing. They rise in approximately 20 minutes, which is sufficient for application to a large dermal wound, yet short enough to cure in a reasonable amount of time. They tighten only minimally after rising. This suggests that they will be suitable for injectable wound healing applications, since they will fill a wound site in a reasonable amount of time and not contract away from the wound boundaries. Their formation by a two-component reactive liquid mixture allows them to be applied easily and conform to the wound boundaries. Their high porosities promote cellular infiltration into the scaffolds and generation of new tissue. Because the strength of the scaffolds is lower than that of native bone, they are particularly suitable for non-weight-bearing sites such as dermal wounds and long-bone fractures.

The invention thus being described, it will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the Specification, including the Example and Attachment be considered as exemplary only, and not intended to limit the scope and spirit of the invention.

Unless otherwise indicated, all numbers expressing quantities of ingredients, properties such as reaction conditions, and so forth used herein are to be understood as being modified in all instances by the term “about.” Accordingly, unless indicated to the contrary, the numerical parameters set forth in the herein are approximations that may vary depending upon the desired properties sought to be determined by the present invention.

Notwithstanding that the numerical ranges and parameters setting forth the broad scope of the invention are approximations, the numerical values set forth in the experimental or example sections are reported as precisely as possible. Any numerical value, however, inherently contain certain errors necessarily resulting from the standard deviation found in their respective testing measurements.

Throughout this application, various publications are referenced. All such references, specifically including those listed below, are incorporated herein by reference.

### REFERENCES

J. S. Temenoff and A. G. Mikos. Injectable biodegradable materials for orthopedic tissue engineering. Biomaterials 21: 2405-2412 (2000).
P. Gunatillake, R. Mayadunne, R. Adhikari, and M. R. El-Gewely. Recent developments in biodegradable synthetic polymers, Biotechnology Annual Review, Vol. Volume 12, Elsevier, 2006, pp. 301-347.
S. L. Bourke and J. Kohn. Polymers derived from the amino acid-tyrosine: polycarbonates, polyarylates and copolymers with poly(ethylene glycol). Advanced Drug Delivery Reviews 55: 447-466 (2003).
S. I. Ertel and J. Kohn. Evaluation of a series of tyrosine-derived polycarbonates as degradable biomaterials. Journal of Biomedical Materials Research 28: 919-930 (1994).
C. Yu and J. Kohn. Tyrosine-PEG-derived poly(ether carbonate)s as new biomaterials: Part 1: synthesis and evaluation. Biomaterials 20: 253-264 (1999).
A. D. Augst, H.-J. Kong, and D. J. Mooney. Alginate Hydrogels as Biomaterials. Macromolecular Bioscience 6: 623-633 (2006).
R. R. Chen and D. J. Mooney. Polymeric growth factor delivery strategies for tissue engineering. Pharmaceutical Research 20: 1103-1112 (2003).
N. Kipshidze, P. Chawla, and M. H. Keelan. Fibrin Meshwork as a Carrier for Delivery of Angiogenic Growth Factors in Patients With Ischemic Limb. Mayo Clinic Proceedings 74: 847-848 (1999).
V. Labhasetwar, J. Bonadio, S. Goldstein, W. Chen, and R. J. Levy. A DNA controlled-release coating for gene transfer: Transfection in skeletal and cardiac muscle. Journal of Pharmaceutical Sciences 87: 1347-1350 (1998).
K. Mizuno, K. Yamamura, K. Yano, T. Osada, S. Saeki, N. Takimoto, T. Sakurai, and Y. Nimura. Effect of chitosan film containing basic fibroblast growth factor on wound healing in genetically diabetic mice. Journal of Biomedical Materials Research Part A 64A: 177-181 (2003).
C. A. Simmons, E. Alsberg, S. Hsiong, W. J. Kim, and D. J. Mooney. Dual growth factor delivery and controlled scaffold degradation enhance in vivo bone formation by transplanted bone marrow stromal cells. Bone 35: 562-569 (2004).
S. A. Zawko, Q. Truong, and C. E. Schmidt. Drug-binding hydrogels of hyaluronic acid functionalized with β-cyclodextrin. Journal of Biomedical Materials Research Part A 9999: NA (2008).
M. D. Timmer, C. G. Ambrose, and A. G. Mikos. Evaluation of thermal- and photo-crosslinked biodegradable poly(propylene fumarate)-based networks. Journal of Biomedical Materials Research Part A 66A: 811-818 (2003).
C. W. Kim, R. Talac, L. Lu, M. J. Moore, B. L. Currier, and M. J. Yaszemski. Characterization of porous injectable poly-(propylene fumarate)-based bone graft substitute. Journal of Biomedical Materials Research Part A 9999: NA (2007).
J. P. Fisher, J. W. M. Vehof, D. Dean, J. P. van der Waerden, T. A. Holland, A. G. Mikos, and J. A. Jansen. Soft and hard tissue response to photocrosslinked poly(propylene fumarate) scaffolds in a rabbit model. Journal of Biomedical Materials Research 59: 547-556 (2002).
S. J. Peter, L. Lu, D. J. Kim, and A. G. Mikos. Marrow stromal osteoblast function on a poly(propylene fumarate)/[beta]-tricalcium phosphate biodegradable orthopaedic composite. Biomaterials 21: 1207-1213 (2000).
S. J. Peter, S. T. Miller, G. Zhu, A. W. Yasko, and A. G. Mikos. In vivo degradation of a poly(propylene fumarate)/β-tricalcium phosphate injectable composite scaffold. Journal of Biomedical Materials Research 41: 1-7 (1998).
M. J. Yaszemski, R. G. Payne, W. C. Hayes, R. S. Langer, T. B. Aufdemorte, and A. G. Mikos. The Ingrowth of New Bone Tissue and Initial Mechanical Properties of a Degrading Polymeric Composite Scaffold. Tissue Engineering 1: 41-52 (1995).
D. H. R. Kempen, L. Lu, C. Kim, X. Zhu, W. J. A. Dhert, B. L. Curreir, and M. J. Yaszemski. Controlled drug release from a novel injectable biodegradable microsphere/scaffold composite based on poly(propylene fumarate). Journal of Biomedical Materials Research, Part A 77A: 103-111 (2006).
I. C. Bonzani, R. Adhikari, S. Houshyar, R. Mayadunne, P. Gunatillake, and M. M. Stevens. Synthesis of two-component injectable polyurethanes for bone tissue engineering. Biomaterials 28: 423-433 (2007).
K. Gorna and S. Gogolewski. Preparation, degradation, and calcification of biodegradable polyurethane foams for bone graft substitutes. Journal of Biomedical Materials Research, Part A 67A: 813-827 (2003).
S. Guelcher, A. Srinivasan, A. Hafeman, K. Gallagher, J. Doctor, S. Khetan, S. McBride, and J. Hollinger. Synthesis, in vitro degradation, and mechanical properties of two-component poly(ester urethane)urea scaffolds: Effects of water and polyol composition. Tissue Engineering 13: 2321-2333 (2007).
J.-Y. Zhang, E. J. Beckman, J. Hu, G.-g. Yang, S. Agarwal, and J. O. Hollinger. Synthesis, biodegradability, and biocompatibility of lysine diisocyanate-glucose polymers. Tissue Engineering 8: 771-785 (2002).
R. Adhikari and P. A. Gunatillake. Biodegradable polyurethane/urea compositions, 2004.
M. Kitai, H. Ryuutou, T. Yahata, Y. Hara, H. Iwane, and Y. Soejima. Aliphatic triisocyanate compound, process for producing the same, and polyurethane resin made from the compound. In U. PTO (ed), Vol. 20020123644 A1 (U. PTO, ed), 2002.
S. A. Guelcher, V. Patel, K. M. Gallagher, S. Connolly, J. E. Didier, J. S. Doctor, and J. O. Hollinger. Synthesis and in vitro biocompatibility of injectable polyurethane foam scaffolds. Tissue Engineering 12: 1247-1259 (2006).
A. S. Sawhney and J. A. Hubbell. Rapidly degraded terpolymers of D,L-lactide, glycolide, and ε-caprolactone with increased hydrophilicity by coploymerization with polyethers. Journal of Biomedical Materials Research 24: 1397-1411 (1990).
ASTM-International. D3574-05. Standard test methods for flexible cellular materials—slab, bonded, and molded urethane foams, Vol. 08.02, 2007, pp. 360-368.
G. Evans, B. Atkinson, and G. Jameson. The Jameson Cell. In K. Matis (ed), Flotation Science and Engineering (K. Matis, ed), Marcel Dekker, New York, 1995, pp. 353.
ASTM-International. D695-02a. Standard Test Method for Compressive Properties of Rigid Plastics, Vol. 14.02, 2007.
G. Oertel. Polyurethane Handbook, Hanser Gardner Publications, Berlin, 1994.
R. Eriksson, T. Albrektsson, and B. Magnusson. Assessment of bone viability after heat trauma. A histological, histochemical and vital microscopic study in the rabbit. Scandinavian Journal of Plastic and Reconstructive Surgery 18: 261-268 (1984).
P. J. Meyer, E. Lautenschlager, and B. Moore. On the setting properties of acrylic bone cement. Journal of Bone & Joint Surgery. American volume 55: 149-156 (1973).
J. E. Mark, E. Erman, and F. R. Eirich. Science and Technology of Rubber, Academic Press, Inc., San Diego, Calif. (1994).
O. Kramer, S. Hvidt, and J. D. Ferry. Dynamic Mechanical Properties. In J. E. Mark, E. Erman, and F. R. Eirich (eds), Science and Technology of Rubber (J. E. Mark, E. Erman, and F. R. Eirich, eds), Academic Press, Inc., San Diego, Calif., 1994.
S. Gogolewski, K. Gorna, and A. S. Turner. Regeneration of bicortical defects in the iliac crest of estrogen-deficient sheep, using new biodegradable polyurethane bone graft substitutes. Journal of Biomedical Materials Research, Part A 77A: 802-810 (2006).
J. Guan, K. L. Fujimoto, M. S. Sacks, and W. R. Wagner. Preparation and characterization of highly porous, biodegradable polyurethane scaffolds for soft tissue applications. Biomaterials 26: 3961-3971 (2005).
J.-Y. Zhang, E. J. Beckman, N. P. Piesco, and S. Agarwal. A new peptide-based urethane polymer: synthesis, biodegradation, and potential to support cell growth in vitro. Biomaterials 21: 1247-1258 (2000).
J. H. de Groot, R. de Vrijer, B. S. Wildeboer, C. S. Spaans, and A. J. Pennings. New biomedical polyurethane ureas with high tear strengths. Polymer Bulletin 38: 211-218 (1997).
G. A. Skarja and K. A. Woodhouse. Structure-property relationships of degradable polyurethane elastomers containing an amino acid-based chain extender. Journal of Applied Polymer Science 75: 1522-1534 (2000).
N. Tsutsumi, S. Yoshizaki, W. Sakai, and T. Kiyotsukuri. Nonlinear optical polymers. 1. Novel network polyurethane with azobenzene dye in the main frame. Macromolecules 28: 6437-6442 (1995).
S. Gogolewski and K. Gorna. Biodegradable polyurethane cancellous bone graft substitutes in the treatment of iliac crest defects. Journal of Biomedical Materials Research Part A 80A: 94-101 (2007).
K. Gorna and S. Gogolewski. Molecular stability, mechanical properties, surface characteristics and sterility of biodegradable polyurethanes treated with low-temperature plasma. Polymer Degradation and Stability 79: 475-485 (2003).
K. Gorna, S. Polowinski, and S. Gogolewski. Synthesis and characterization of biodegradable poly(δ-caprolactone urethane)s. I. Effect of the polyol molecular weight, catalyst, and chain extender on the molecular and physical characteristics. Journal of Polymer Science, Part A: Polymer Chemistry 40: 156-170 (2002).
J. Guan, M. S. Sacks, E. J. Beckman, and W. R. Wagner. Biodegradable poly(ether ester urethane)urea elastomers based on poly(ether ester) triblock copolymers and putrescine: synthesis, characterization and cytocompatibility. Biomaterials 25: 85-96 (2004).
P. Bruin, G. J. Veenstra, A. J. Nijenhuis, and A. J. Pennings. Design and synthesis of biodegradable poly(ester-urethane) elastomer networks composed of non-toxic building blocks. Die Makromolekulare Chemie, Rapid Communications 9: 589-594 (1988).
S. L. Elliott, J. D. Fromstein, J. P. Santerre, and K. A. Woodhouse. Identification of biodegradation products formed by L-phenylalanine based segmented polyurethaneureas. Journal of Biomaterials Science Polymer Edition. 13: 691-711 (2002).
L. Lu, S. J. Peter, M. D. Lyman, H.-L. Lai, S. M. Leite, J. A. Tamada, S. Uyama, J. P. Vacanti, R. Langer, and A. G. Mikos. In vitro and in vivo degradation of porous poly(D-lactic-co-glycolic acid) foams. Biomaterials 21: 1837-1845 (2000).
J. Guan, J. J. Stankus, and W. R. Wagner. Biodegradable elastomeric scaffolds with basic fibroblast growth factor release. Journal of Controlled Release 120: 70-78 (2007).
J.-Y. Zhang, B. A. Doll, E. J. Beckman, and J. O. Hollinger. A biodegradable polyurethane-ascorbic acid scaffold for bone tissue engineering. Journal of Biomedical Materials Research, Part A 67: 389-400 (2003).

We claim:

1. A method of synthesizing of a biocompatible and biodegradable polyurethane foam comprising the steps of:
mixing at least one biocompatible polyol, PEG, water, at least one stabilizer, and at least one pore opener, to form a resin mix;
contacting the resin mix with at least one HDIt polyisocyanate to form a reactive liquid mixture; and
reacting the reactive liquid mixture form a polyurethane foam; the polyurethane foam being biodegradable within a living organism to biocompatible degradation products.

2. The method of claim 1 wherein at least one catalyst is added to form the resin mix.

3. The method of claim 1, wherein the PEG is MW 600.

4. The method of claim 1, wherein the mixing step comprises mixing a catalyst, stabilizer, and pore opener.

5. The method of claim 4, wherein the catalyst is a triethylenediamine catalyst.

6. The method of claim 5, where in the stabilizer is a sulfated castor oil stabilizer.

7. The method of claim 4, wherein the pore opener is a calcium stearate cell opener.

8. The method of claim 1, wherein the PEG is added in an amount up to about 60% polyol component.

9. A biodegradable polyurethane scaffold, comprising
HDI trimer polyisocyanate;
at least one polyol; wherein
the density of said scaffold is from about 50 to about 250 kg m-3 and the porosity of the scaffold is greater than about 70 (vol %) and at least 50% of the pores are interconnected with another pore.

10. The polyurethane scaffold of claim 9, wherein the density is at least 90 kg m-3.

11. The polyurethane scaffold of claim 9, wherein the density is from about 75 to about 125 kg m-3.

12. The polyurethane scaffold of claim 9, further comprising PEG.

13. The polyurethane scaffold of claim 12, wherein the PEG is present in an amount of about 50% or less w/w.

14. The polyurethane scaffold of claim 13, wherein the PEG is present in an amount of about 30% or less w/w.

15. The polyurethane scaffold of claim 9, wherein the glass transition temperature is in a range of about −50 to about 20.

16. The polyurethane scaffold of claim 15, wherein the glass transition temperature is in a range of about −20 to about 10.

17. The polyurethane scaffold of claim 9, wherein the porosity is greater than 70 (vol-%).

18. The polyurethane scaffold of claim 17, wherein the porosity is from about 90 to about 95 (vol-%).

19. The polyurethane scaffold of claim 9, wherein the pore size is about 100-1000 μm.

20. The polyurethane scaffold of claim 9, wherein the pore size is about 200-500 μm.

21. The polyurethane scaffold of claim 9, further comprising at least one growth factor.

22. The polyurethane scaffold of claim 21, wherein the growth factor is chosen from PDGF, VEGF, and BMP-2.

23. The polyurethane scaffold of claim 9, further comprising a stabilizer chosen from a polyethersiloxane, sulfonated caster oil, and sodium ricinoleicsulfonate.

24. The polyurethane scaffold of claim 9, further comprising a biologically active agent.

25. The polyurethane scaffold of claim 24, wherein the biologically active agent comprises demineralized bone particles.

26. The polyurethane scaffold of claim 24, wherein the biologically active agent is chosen from enzymes, organic catalysts, ribozymes, organometallics, proteins, glycoproteins, peptides, polyamino acids, antibodies, nucleic acids, steroidal molecules, antibiotics, antivirals, antimycotics, anticancer agents, analgesic agents, antirejection agents, immunosuppressants, cytokines, carbohydrates, oleophobics, lipids, extracellular matrix and/or its individual components, demineralized bone matrix, pharmaceuticals, chemotherapeutics, cells, viruses, virenos, virus vectors, and prions.

27. The polyurethane scaffold of claim 9, wherein the HDI trimer is present in an amount of from about 30 to about 75 wt %.

28. The polyurethane scaffold of claim 9, wherein the HDI trimer is present in an amount of from about 40 to about 70 wt %.

29. The polyurethane scaffold of claim 9, wherein the polyol is a polyester triol present in an amount of from about 10 to about 70 wt %.

30. The polyurethane scaffold of claim 9, wherein the polyol is a polyester triol present in an amount of from about 20 to about 60 wt %.

31. The polyurethane scaffold of claim 12, wherein the PEG is present in an amount of about 40 wt % or less.

32. The polyurethane scaffold of claim 12, wherein the PEG is present in an amount of about 30 wt % or less.

33. The polyurethane scaffold of claim 9, wherein the permanent deformation of the scaffold is less than about 3.0%.

34. A biodegradable polyurethane scaffold, comprising
HDI trimer polyisocyanate in an amount of from about 40 to about 70 wt %;
a polyester triol present in an amount of from about 20 to about 60 wt %;
PEG in an amount of from about 30 wt % or less; wherein
the permanent deformation of the scaffold is less than about 3.0%.
